(19) United States
(12) Patent Application Publication
(10) Pub. No.: US 2026/0150838 A1
(43) Pub. Date: Jun. 4, 2026

(54) ASCAROSIDE DERIVATIVES

(71) Applicant: Ascribe Bioscience Inc., Ithaca, NY (US)

(72) Inventors: Jay Farmer, Ithaca, NY (US); Uwe Conrath, Aachen, North Rhine-Westphalia (DE)

(21) Appl. No.: 19/156,948

(22) PCT Filed: Feb. 20, 2024
(86) PCT No.: PCT/US2024/016565

§ 371 Date: Aug. 15, 2025

Related U.S. Application Data
(60) Provisional application No. 63/486,019, filed on Feb. 20, 2023.

(51) Int. Cl. C07D 401/12

(52) U.S. Cl. CPC A01N 43/16; A01P 3/00; A61K 31/7028

(57) ABSTRACT

This application relates to derivatives of ascarosides containing nitrogen- or sulfur-containing functional groups. The application further relates to methods of using such derivatives of ascarosides.

### CROSS-REFERENCE TO RELATED APPLICATIONS

This application claims priority to U.S. Provisional Patent Application Ser. No. 63/486,019 filed on Feb. 20, 2023, the entirety of which is incorporated herein by reference.

### FIELD OF THE INVENTION

This application generally relates to derivatives of biologically active compounds, compositions and to methods of treating animals or plants with such compounds to promote health, ameliorate disease, and/or enhance resistance to pathogens.

### BACKGROUND OF THE INVENTION

Ascaroside natural products are secondary metabolites produced by nematodes. A large number of structurally diverse ascaroside structures have been identified in nature and the molecules are believed to function as an evolutionarily conserved chemical language used by nematodes to control many aspects of their development. Ascarosides are also perceived by other organisms and have been demonstrated to have a range of effects on numerous non-nematode organisms including bacteria, fungi, plants, and mammals including humans. Ascarosides hold potential as human medicines, agrichemicals, and products for other diverse and valuable applications.

Ascarosides have been demonstrated to enhance plant growth and to increase plant resistance to certain pathogens and/or induce the priming of plant defense responses (which can inhibit pathogenic growth and/or infestation) when applied to the plant. By activating and/or priming plants' innate defenses, ascarosides can thereby prevent proliferation of pathogens.

It would be useful to provide ascarosides in forms that enhance their uptake, half-life and/or biological activities and/or to modify the physical properties of the compounds to make their formulation or application easier and, further, to provide additional benefits to plants or other organisms to which such modified ascarosides are applied or administered.

### SUMMARY OF THE INVENTION

The disclosure provides compounds, compositions, and methods for ascaroside delivery. In certain embodiments, such compounds, compositions and methods are particularly useful in an agrichemical context and the disclosure primarily describes such uses—however, it is also recognized that the ascaroside derivatives and related formulations and methods described herein may have applications beyond agriculture—for example as therapeutics to improve the health of humans or other animals. The compositions and methods herein relate generally to derivatives of ascarosides which, in some embodiments provide various benefits when administered or applied to an organism (e.g., to a plant, an animal, or a microbe). In general, naturally occurring ascarosides comprise a fatty acid sidechain with a terminal carboxylic acid or ester group:

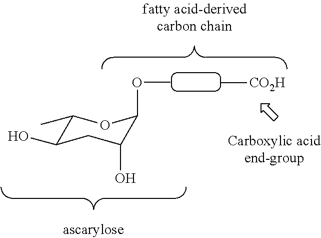

### Structure of a Typical Natural Ascaroside

In certain embodiments, ascaroside derivatives provided by the present invention comprise a nitrogen- or sulfur-containing functional group in place of the typical carboxylic acid (or ester) end group found on the sidechain of natural ascarosides.

In certain embodiments, ascaroside derivatives provided by the present invention comprise a nitrogen-containing functional group. In certain embodiments, such derivatives feature an amide, an amidine, an imide, or a nitrile functional group. In certain embodiments, ascaroside derivatives provided by the present invention comprise a sulfur-containing functional group. In certain embodiments, such derivatives feature a thioacid, a thioester, a thioamide, or a thioimide functional group. In certain embodiments, provided derivatives comprise a dimer or oligomer of two or more ascaroside molecules linked through a polyfunctional moiety comprising one or more nitrogen- and/or sulfur-containing functional groups.

In certain embodiments, provided ascaroside derivatives are characterized in that their uptake by an organism is enhanced relative to a corresponding ascaroside carboxylic acid or ester. In certain embodiments, the provided ascaroside derivatives are characterized in that they hydrolyze after uptake by an organism to liberate the corresponding ascaroside carboxylic acid. In certain embodiments, such ascaroside derivatives are characterized in that they hydrolyze after application (e.g. after applying to surface of a plant or organism or the immediate environment of such a plant or organism) to liberate the corresponding ascaroside carboxylic acid. In certain embodiments, provided ascaroside derivatives are biologically active molecules aside from, or in addition to, their activity as ascaroside precursors.

The disclosure includes, without limitations, the following embodiments.

Embodiment 1: A composition comprising an ascaroside derivative of formula I:

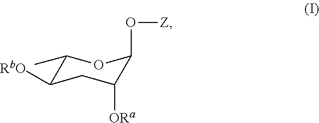

wherein: each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g.—a moiety —C(O)OR^{c}), a C₂₋₂₀ carbamate (e.g.—a moiety —C(O)N(R^{c})₂), a C₂₋₂₀ thioester (e.g. a moiety —C(S)R^{c}), a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety —C(S)N(R^{c})₂), a sugar moiety, a peptide, a polymer chain, or a linkage via a bond or a carbon-containing linker moiety to another ascaroside molecule; where Re is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule; and where R^{a} and R^{b} may be taken together to form an optionally substituted ring, optionally containing one or more heteroatoms, and optionally containing one or more sites of unsaturation; and −Z comprises an optionally unsaturated, optionally substituted C₂₋₄₀ sidechain terminating in a nitrogen- or sulfur-containing functional group.

Embodiment 2. The composition of Embodiment 1, wherein —Z has a structure selected from the group consisting of: —CH(CH₃)—(CH₂)ₙ—Q, —CH(CH₃)—(CH₂)ₙ—CH═CH—Q, —CH(CH₃)—(CH₂)ₙ—CH═CH—(CH₂)ₙ—Q, —CH(CH₃)—(CH₂)ₙ—CH(OH)—CH₂—Q, —CH(CH₃)—(CH₂)ₙ—CH(OH)—Q, —(CH₂)ₙ—Q, —(CH₂)ₙ—CH═CH—Q, —(CH₂)ₙ—CH═CH—(CH₂)ₙ—Q, —(CH₂)ₙ—CH(OH)—CH₂—Q, and —(CH₂)ₙ—CH(OH)—Q, where —Q is a nitrogen- or sulfur-containing functional group, and n is independently at each occurrence an integer between 1 and 40.

Embodiment 3: The composition of Embodiment 2, wherein —Q is a nitrogen-containing functional group.

Embodiment 4: The composition of Embodiment 3, wherein Q is selected from the group consisting of: amide, amidine, nitrile and imide.

Embodiment 5: The composition of Embodiment 3, wherein —Q is selected from the group consisting of: —C(O)NR¹R², —C(NR⁴)NR¹R², —C(O)NR¹C(O)R³, and —C≡N, where: at each occurrence, R¹, R², and R³ are selected independently from the group consisting of: —H, optionally substituted C₁₋₁₂ aliphatic, optionally substituted C₁₋₁₂ heteroaliphatic, optionally substituted aryl, optionally substituted heteroaryl, a polymer chain, and a linkage via a bond or a carbon-containing linker moiety to another ascaroside molecule; where R¹ and R², or R¹ and R³ may optionally be taken together to form an optionally substituted ring, optionally containing one or more heteroatoms and optionally containing one or more sites of unsaturation; R⁴ is selected from the group consisting of: —H, optionally substituted C₁₋₁₂ aliphatic, optionally substituted C₁₋₁₂ heteroaliphatic, optionally substituted aryl, optionally substituted heteroaryl, and a polymer chain, or a linkage via a bond or a carbon-containing linker moiety to another ascaroside molecule.

Embodiment 6: The composition of Embodiment 2, wherein —Q is a sulfur-containing functional group.

Embodiment 7: The composition of Embodiment 6, wherein Q is selected from the group consisting of: thioacid, thioester, dithioester, thioamide, and thioimide.

Embodiment 8: The composition of Embodiment 6, wherein —Q is selected from the group consisting of —C(S)OR¹, —C(O)—S, —SR¹, —C(S)—Sₓ—SR¹, —C(S)NR¹R², —C(S)NR¹C(O)R³, —C(O)NR¹C(S)R³, and —C(S)NR¹C(S)R³, where: x is an integer between 0 and 6, and at each occurrence; and R¹, R², and R³ are selected independently from the group consisting of: —H, optionally substituted C₁₋₁₂ aliphatic, optionally substituted C₁₋₁₂ heteroaliphatic, optionally substituted aryl, optionally substituted heteroaryl, a polymer chain, and a linkage via a bond or a carbon-containing linker moiety to another ascaroside molecule: where R¹ and R², or R¹ and R³ may optionally be taken together to form an optionally substituted ring, optionally containing one or more heteroatoms and optionally containing one or more sites of unsaturation.

Embodiment 9: The composition of any of Embodiments 1-8, further comprising one or more additional components selected from the group consisting of surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, and wetting agents.

Embodiment 10: A method of treating an organism with an ascaroside, comprising contacting the organism with one or more ascaroside derivatives containing a nitrogen- or sulfur-containing functional group, wherein the nitrogen- or sulfur-containing functional group is then hydrolyzed or metabolized to a carboxylic acid (or an ester or salt thereof).

Embodiment 11: The method of Embodiment 10, wherein the one or more ascaroside derivatives are within a composition.

Embodiment 12: A method of treating an organism with an ascaroside, comprising contacting the organism with a composition according to any of Embodiments 1-9.

Embodiment 13: The method of any of Embodiments 10-12, wherein the organism is a plant.

Embodiment 12: The method of any of Embodiments 10-12, wherein the organism is a human.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present disclosure will become apparent from the following.

### BRIEF DESCRIPTION OF THE DRAWING

Having thus described aspects of the disclosure in the foregoing general terms, reference will now be made to the accompanying drawing: the drawing is exemplary only, and should not be construed as limiting the disclosure.

FIG. 1 is a bar chart of data on reduction of fungal infection effected by certain non-limiting ascaroside derivatives according to some embodiments of the present disclosure.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. As used in this application, the term “comprise” and variations of the term, such as “comprising” and “comprises,” are not intended to exclude other additives, components, integers or steps.

About, Approximately: As used herein, the terms “about” and “approximately” are used as equivalents. Unless otherwise stated, the terms “about” and “approximately” may be understood to permit standard variation as would be understood by those of ordinary skill in the art. Where ranges are provided herein, the endpoints are included. Any numerals used in this application with or without about/approximately are meant to cover any normal fluctuations appreciated by one of ordinary skill in the relevant art. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books. Sausalito, 1999; Smith and March March's Advanced Organic Chemistry, 5^{th} Edition, John Wiley & Sons, Inc., New York, 2001: Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

Certain derivatives provided herein can comprise one or more asymmetric centers, and thus can exist in various stereoisomeric forms, e.g., enantiomers and/or diastereomers. Thus, inventive ascaroside derivatives and compositions thereof may be in the form of an individual enantiomer, diastereomer or geometric isomer, or may be in the form of a mixture of stereoisomers. In certain embodiments, compounds described herein are enantiopure compounds. In certain other embodiments, mixtures of enantiomers or diastereomers are provided.

Furthermore, certain ascaroside derivatives as described herein may have one or more double bonds that can exist as either a Z or E isomer, unless otherwise indicated. The compounds can be provided as individual isomers substantially free of other isomers and alternatively, as mixtures of various isomers, e.g., racemic mixtures of enantiomers.

As used herein, the term “isomers” includes any and all geometric isomers and stereoisomers. For example, “isomers” include cis- and trans-isomers, E- and Z-isomers, R- and S-enantiomers, diastereomers, (D)-isomers, (L)-isomers, racemic mixtures thereof, and other mixtures thereof, as falling within the scope of the disclosure. For instance, a compound may, in some embodiments, be provided substantially free of one or more corresponding stereoisomers, and may also be referred to as “stereochemically enriched.”

Where a particular enantiomer is preferred, it may, in some embodiments be provided substantially free of the opposite enantiomer, and may also be referred to as “optically enriched.” “Optically enriched,” as used herein, means that the compound is made up of a significantly greater proportion of one enantiomer. In certain embodiments the compound is made up of at least about 90% by weight of an enantiomer. In some embodiments the compound is made up of at least about 95%, 97%, 98%, 99%, 99.5%, 99.7%, 99.8%, or 99.9% by weight of an enantiomer. In some embodiments the enantiomeric excess of provided compounds is at least about 90%, 95%, 97%, 98%, 99%, 99.5%, 99.7%, 99.8%, or 99.9%. In some embodiments, enantiomers may be isolated from racemic mixtures by any method known to those skilled in the art, including chiral high pressure liquid chromatography (HPLC) and the formation and crystallization of chiral salts or prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972).

The terms “halo” and “halogen” as used herein refer to an atom selected from fluorine (fluoro, —F), chlorine (chloro, —Cl), bromine (bromo, —Br), and iodine (iodo, —I).

The term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-30 carbon atoms. In certain embodiments, aliphatic groups contain 1-12 carbon atoms. In certain embodiments, aliphatic groups contain 1-8 carbon atoms. In certain embodiments, aliphatic groups contain 1-6 carbon atoms. In some embodiments, aliphatic groups contain 1-5 carbon atoms, in some embodiments, aliphatic groups contain 1-4 carbon atoms, in yet other embodiments aliphatic groups contain 1-3 carbon atoms, and in yet other embodiments aliphatic groups contain 1-2 carbon atoms. Suitable aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “heteroaliphatic” or “heteroaliphatic group”, as used herein, denotes an aliphatic group where one or more carbon or hydrogen atoms are replaced by a heteroatom (e.g. oxygen, nitrogen, sulfur, phosphorous, boron, etc.).

The term “unsaturated”, as used herein, means that a moiety has one or more double or triple bonds.

The term “alkyl,” as used herein, refers to saturated, straight- or branched-chain hydrocarbon radicals derived from an aliphatic moiety containing between one and six carbon atoms by removal of a single hydrogen atom. Unless otherwise specified, alkyl groups contain 1-12 carbon atoms. In certain embodiments, alkyl groups contain 1-8 carbon atoms. In certain embodiments, alkyl groups contain 1-6 carbon atoms. In some embodiments, alkyl groups contain 1-5 carbon atoms, in some embodiments, alkyl groups contain 1-4 carbon atoms, in yet other embodiments alkyl groups contain 1-3 carbon atoms, and in yet other embodiments alkyl groups contain 1-2 carbon atoms. Examples of alkyl radicals include, but are not limited to, methyl, ethyl, n propyl, isopropyl, n butyl, iso butyl, sec butyl, sec pentyl, iso pentyl, tert butyl, n-pentyl, neopentyl, n-hexyl, sec-hexyl, n-heptyl, n-octyl, n-decyl, n-undecyl, dodecyl, and the like.

The term “alkenyl,” as used herein, denotes a monovalent group derived from a straight- or branched-chain aliphatic moiety having at least one carbon-carbon double bond by the removal of a single hydrogen atom. Unless otherwise specified, alkenyl groups contain 2-12 carbon atoms. In certain embodiments, alkenyl groups contain 2-8 carbon atoms. In certain embodiments, alkenyl groups contain 2-6 carbon atoms. In some embodiments, alkenyl groups contain 2-5 carbon atoms, in some embodiments, alkenyl groups contain 2-4 carbon atoms, in yet other embodiments alkenyl groups contain 2-3 carbon atoms, and in yet other embodiments alkenyl groups contain 2 carbon atoms. Alkenyl groups include, for example, ethenyl, propenyl, butenyl, 1-methyl-2-buten-1-yl, and the like.

The term “aryl” used alone or as part of a larger moiety as in “aralkyl”, “aralkoxy”, or “aryloxyalkyl”, refers to monocyclic and polycyclic ring systems having a total of five to 20 ring members, wherein at least one ring in the system is aromatic and wherein each ring in the system contains three to twelve ring members. The term “aryl” may be used interchangeably with the term “aryl ring”. In certain embodiments, “aryl” refers to an aromatic ring system which includes, but is not limited to, phenyl, biphenyl, naphthyl, anthracyl and the like, which may bear one or more substituents. Also included within the scope of the term “aryl”, as it is used herein, is a group in which an aromatic ring is fused to one or more additional rings, such as benzofuranyl, indanyl, phthalimidyl, naphthimidyl, phenantriidinyl, or tetrahydronaphthyl, and the like.

As described herein, ascaroside derivatives as provided herein may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position.

Combinations of substituents envisioned are preferably those that result in the formation of stable or chemically feasible compounds/derivatives. The term “stable.” as used herein, refers to compounds or derivatives that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen: —(CH₂)₀₋₄R^{◯}; —(CH₂)₀₋₄OR^{◯}; —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —NO₂; —CN; —N₃; —(CH₂)₀₋₄N(R^{◯})₂; —(CH₂)₀₋₄N(R^{◯})C(O)R^{◯}; —N(R^{◯})C(S)R^{◯}; —(CH₂)₀₋₄N(R^{◯})C(O)NR^{◯}₂; —N(R^{◯})C(S)NR^{◯}₂; (CH₂)₀₋₄N(R^{◯})C(O)OR^{◯}; —N(R^{◯})N(R^{◯})C(O)R^{◯}; —N(R^{◯})N(R^{◯})C(O)NR^{◯}₂: —N(R^{◯})N(R^{◯})C(O)OR^{◯}; —(CH₂)₀₋₄C(O)R^{◯}; —C(S)R^{◯}; —(CH₂)₀₋₄C(O)OR^{◯}; —(CH₂)₀₋₄C(O)N(R^{◯})₂; —(CH₂)₀₋₄C(O)SR^{◯}; —(CH₂)₀₋₄C(O)OSiR^{◯}₃; —(CH₂)₀₋₄OC(O)R^{◯}; —OC(O)(CH₂)₀₋₄SR—, SC(S)SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, —(CH₂)₀₋₄OC(O)NR^{◯}₂; —C(O)N(OR^{◯})R^{◯}; —C(O)C(O)R^{◯}; —C(O)CH₂C(O)R^{◯}; —C(NOR^{◯})R^{◯}; —(CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; —(CH₂)₀₋₄S(O)R^{◯}; —N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; —P(O)R^{◯}₂; —OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene)C(O)O—N(R^{◯})₂, wherein each R^{◯} may be substituted as defined below and is independently hydrogen, C₁₋₈ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3-12-membered saturated, partially unsaturated, or aryl mono- or polycyclic ring having 0-4 heteroatoms independently selected from nitrogen, oxygen, or sulfur, which may be substituted as defined below:

Suitable monovalent substituents on R^{◯} (or the ring formed by taking two independent occurrences of R^{◯} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₄C(O)N(R^{•})₂; —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{•} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “substantially” refers to the qualitative condition of exhibiting total or near-total extent or degree of a characteristic or property of interest.

The convention of naming ascarosides by a several-letter prefix followed by a pound sign (#) and a number is sometimes used (for example ascr #18). This convention is used in the scientific literature and the skilled artisan will understand that each such name is associated with a specific chemical structure of known composition and will readily apprehend the structure of the molecule referred to using this naming convention. Unless otherwise indicated, all compound identifiers of this format used herein conform to the definitions described in the C. elegans Small Molecule Identifier Database (SMID-DB) maintained at http://www.smid-db.org, the current version of which is hereby incorporated herein by reference.

The term “pathogen” refers to any bacterium, fungus, oomycete, virus, nematode (e.g., cyst or root knot nematode) or insect with pathogenic effects on a plant.

### DETAILED DESCRIPTION

Compounds, compositions, and methods for the use of ascaroside derivatives are provided. The disclosure is directed to ascaroside derivatives, as well as to compositions and methods involving such ascaroside derivatives. In certain embodiments, provided compounds, compositions, and methods herein relate generally to derivatives of ascarosides which, in some embodiments provide various benefits when administered or applied to an organism (e.g., a plant, an animal, or a microbe). In general, ascarosides comprise a fatty acid-derived sidechain (linked to ascarylose via a glycosidic bond) and having a carboxylic acid or ester group at the chain end. In one aspect, the present invention provides ascaroside derivatives that comprise a nitrogen- or sulfur-containing functional group in place of the sidechain's carboxylic acid or ester group.

### I. ASCAROSIDE DERIVATIVES

In one aspect, the present invention provides ascaroside derivatives containing a nitrogen- or sulfur-containing functional group in place of the carboxylic acid or ester typical of ascaroside sidechains. In certain embodiments, such derivatives are novel compounds that have not previously been synthesized or described. In certain embodiments, such derivatives may have been previously known or described, but their utility for formulation of compositions with enhanced utility for the treatment of plants or animals has not been reported or recognized and/or they have not been formulated into defined compositions with utility in such applications.

In certain embodiments, provided ascaroside derivatives conform to Formula-I:

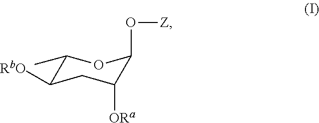

wherein:
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g., a moiety —C(O)OR^{c}), a C₂₋₂₀ carbamate (e.g., -a moiety —C(O)N(R^{c})₂), a C₂₋₂₀ thioester (e.g., a moiety —C(S)R^{c}), a C₂₋₂₀ thiocarbonate (e.g., a moiety —C(S)OR^{c}), a C₂₋₂₀ dithiocarbonate (e.g., a moiety —C(S)SR^{c}), a C₁₋₂₀ thiocarbamate (e.g., a moiety —C(S)N(R^{c})₂), a sugar moiety, a peptide, a polymer chain, or a linkage via a bond or a carbon-containing linker moiety to another ascaroside molecule, where Re is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule; and where R^{a} and R^{b} may be taken together to form an optionally substituted ring, optionally containing one or more heteroatoms, and optionally containing one or more sites of unsaturation; and
—Z comprises an optionally unsaturated, optionally substituted C₂₋₄₀ sidechain terminating in a nitrogen- or sulfur-containing functional group.

In certain embodiments, —Z has a structure selected from the group consisting of: —CH(CH₃)—(CH₂)ₙ—Q, —CH(CH₃)—(CH₂)ₙ—CH═CH—Q, —CH(CH₃)—(CH₂)ₙ—CH═CH—(CH₂)ₙ—Q, —CH(CH₃)—(CH₂)ₙ—CH(OH)—CH₂—Q, —CH(CH₃)—(CH₂)ₙ—CH(OH)—Q, —(CH₂)ₙ—Q, —(CH₂)ₙ—CH═CH—Q, —(CH₂)ₙ—CH═CH—(CH₂)ₙ—Q, —(CH₂)ₙ—CH(OH)—CH₂—Q, and —(CH₂)ₙ—CH(OH)—Q, where —Q is a nitrogen- or sulfur-containing functional group, and n is independently at each occurrence an integer between 1 and 40.

### A. Nitrogen-Containing Ascaroside Derivatives

In certain embodiments, —Q is a nitrogen-containing functional group. In certain embodiments, —Z comprises an optionally unsaturated, optionally substituted C₂₋₄₀ carbon chain terminating in a nitrogen-containing functional group. In certain embodiments, —Z comprises an optionally unsaturated, optionally substituted C₂₋₄₀ carbon chain terminating in a functional group selected from the group consisting of: amide, amidine, nitrile and imide. In certain embodiments, —Z terminates in an amide functional group. In certain embodiments, —Z terminates in an amidine functional group. In certain embodiments, —Z terminates in a nitrile functional group. In certain embodiments, Z terminates in an imide functional group.

In certain embodiments, —Q is selected from the group consisting of: —C(O)NR¹R², —C(NR⁴)NR¹R², —C(O)NR¹C(O)R³, and —C≡N where: at each occurrence, R¹, R², and R³ are selected independently from the group consisting of: —H, optionally substituted C₁₋₁₂ aliphatic, optionally substituted C₁₋₁₂ heteroaliphatic, optionally substituted aryl, optionally substituted heteroaryl, a polymer chain, and a linkage via a bond or a carbon-containing linker moiety to another ascaroside molecule: where R¹ and R², or R¹ and R³ may optionally be taken together to form an optionally substituted ring, optionally containing one or more heteroatoms and optionally containing one or more sites of unsaturation; and R⁴ is selected from the group consisting of: —H, optionally substituted C₁₋₁₂ aliphatic, optionally substituted C₁₋₁₂ heteroaliphatic, optionally substituted aryl, optionally substituted heteroaryl, and a polymer chain, or a linkage via a bond or a carbon-containing linker moiety to another ascaroside molecule.

In certain embodiments, —Q is a nitrogen-containing functional group and at least one of R¹ and R² is —H. In certain embodiments, both R¹ and R² groups are —H. In certain embodiments, —Q is a nitrogen-containing functional group and at least one of R¹ and R² is an optionally substituted C₁₋₂₀ aliphatic group. In certain embodiments, —Q is a nitrogen-containing functional group and both R¹ and R² are an optionally substituted C₁₋₂₀ aliphatic group which may be the same or different. In certain embodiments, —Q is a nitrogen-containing functional group and at least one of R¹ and R² is an optionally substituted C₁₋₁₂ aliphatic group. In certain embodiments, —Q is a nitrogen-containing functional group and at least one of R¹ and R² is an optionally substituted C₁₋₈ aliphatic group. In certain embodiments, —Q is a nitrogen-containing functional group and at least one of R¹ and R² is an optionally substituted C₁₋₆ aliphatic group. In certain embodiments, —Q is a nitrogen-containing functional group and at least one of R¹ and R² is selected from methyl, ethyl, n-propyl, i-propyl, n-butyl, sec-butyl, and t-butyl. In certain embodiments, —Q is a nitrogen-containing functional group and at least one of R¹ and R² is —CH₂CH₂OH. In certain embodiments, —Q is a nitrogen-containing functional group and at least one of R¹ and R² is —CH₂CH₂OR³, where R³ is as defined above and in the genera and subgenera herein. In certain embodiments, —Q is a nitrogen-containing functional group and at least one of R¹ and R² is an optionally substituted aromatic group. In certain embodiments, —Q is a nitrogen-containing functional group and at least one of R¹ and R² comprises a glycoside. In certain embodiments, at least one of R¹ and R² comprises an amino acid. In certain embodiments, —Q is a nitrogen-containing functional group and at least one of R¹ and R² comprises a peptide. In certain embodiments, —Q is a nitrogen-containing functional group and at least one of R¹ and R² comprises a nucleotide.

In certain embodiments, an ascaroside derivative is selected from the group consisting of:

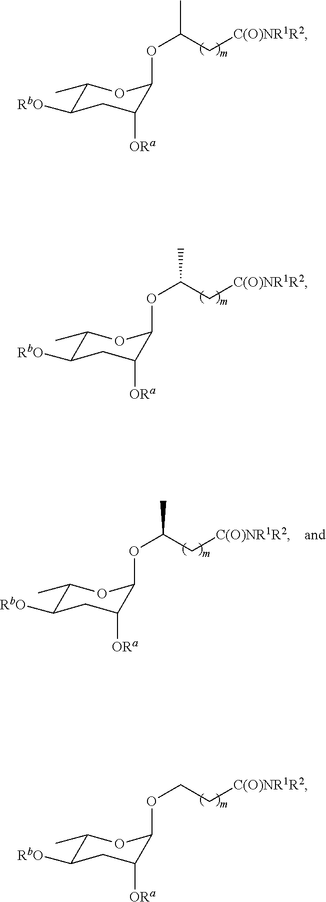

where m is an integer from 1 to 24, and each of R^{a}, R^{b}, R¹ and R² is as defined above and in the genera and subgenera herein.

In certain embodiments, an ascaroside derivative is selected from the group consisting of:

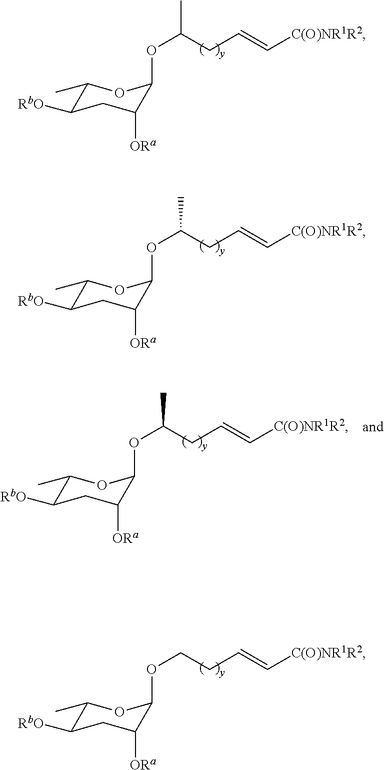

where y is an integer from 1 to 24, and each of R^{a}, R^{b}, R¹ and R² is as defined above and in the genera and subgenera herein.

In certain embodiments, an ascaroside derivative is selected from the group consisting of:

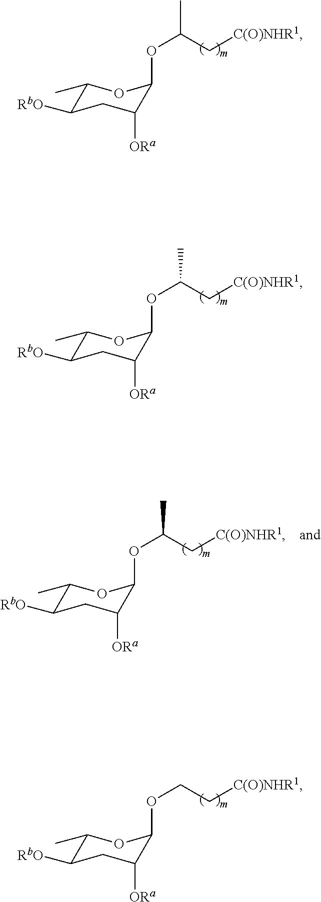

where each of R^{a}, R^{b}, R¹ and m is as defined above and in the genera and subgenera herein.

In certain embodiments, an ascaroside derivative is selected from the group consisting of:

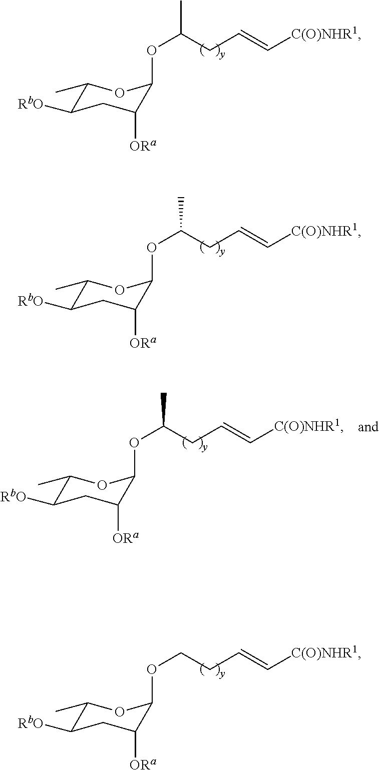

where each of R^{a}, R^{b}, R¹ and y is as defined above and in the genera and subgenera herein.

In certain embodiments, an ascaroside derivative is selected from the group consisting of:

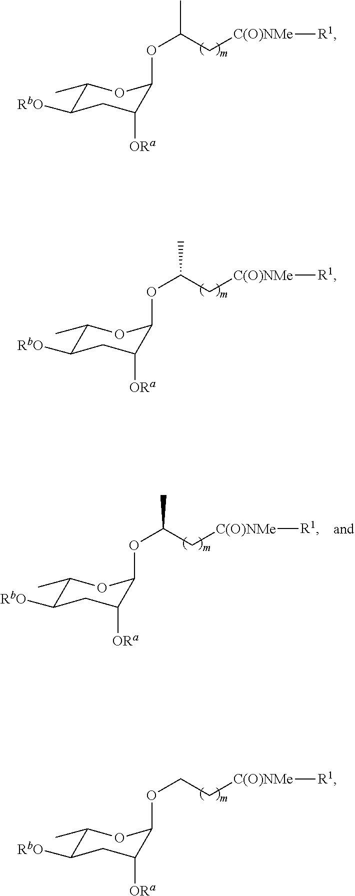

where each of R^{a}, R^{b}, and m is as defined above and in the genera and subgenera herein.

In certain embodiments, an ascaroside derivative is selected from the group consisting of:

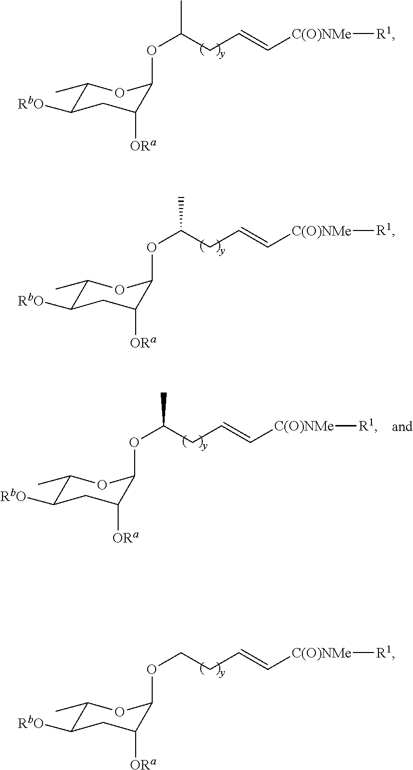

where each of R^{a}, R^{b}, and y is as defined above and in the genera and subgenera herein.

In certain embodiments, an ascaroside derivative is selected from the group consisting of:

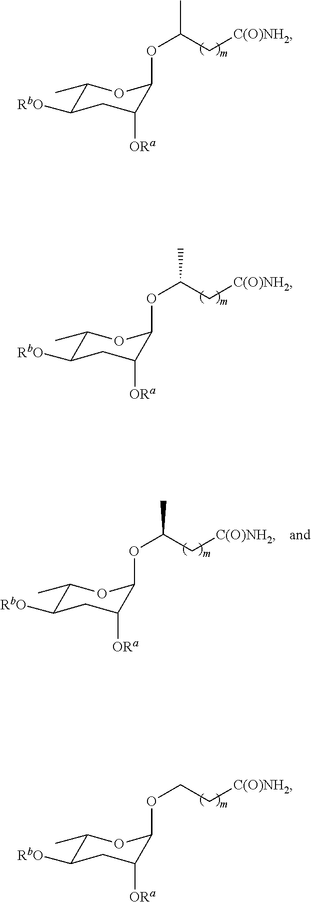

where each of R^{a}, R^{b}, and m is as defined above and in the genera and subgenera herein.

In certain embodiments, an ascaroside derivative is selected from the group consisting of:

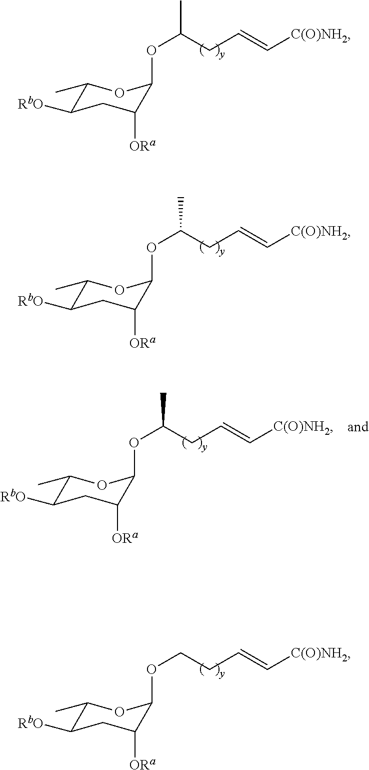

where each of R^{a}, R^{b}, and y is as defined above and in the genera and subgenera herein.

In certain embodiments, an ascaroside derivative is selected from the group consisting of:

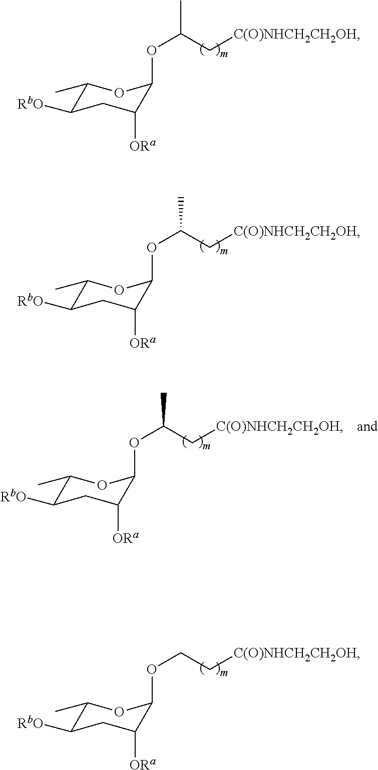

where each of R^{a}, R^{b}, and m is as defined above and in the genera and subgenera herein.

In certain embodiments, an ascaroside derivative is selected from the group consisting of:

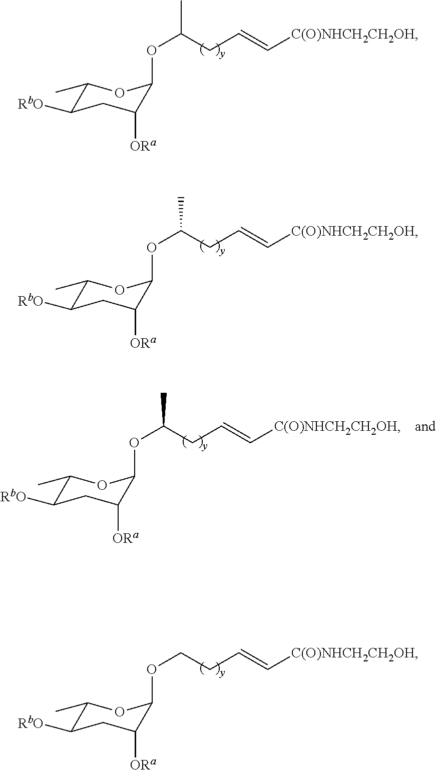

where each of R^{a}, R^{b}, and y is as defined above and in the genera and subgenera herein.

In certain embodiments, an ascaroside derivative is selected from the group consisting of:

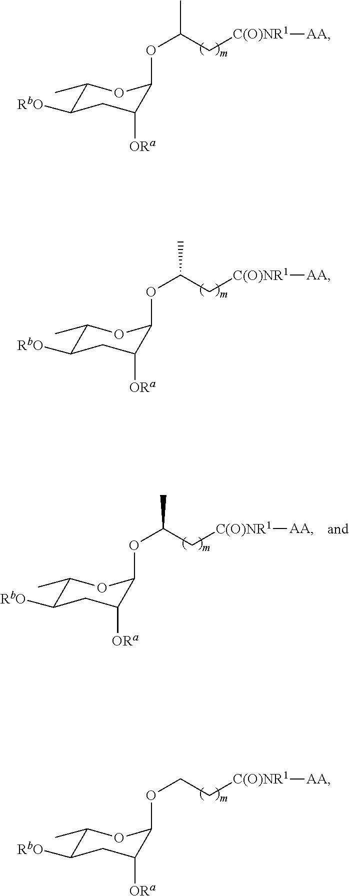

where each of R^{a}, R^{b}, R¹ and m is as defined above and in the genera and subgenera herein, and the moiety -AA is an amino acid or peptide linked through its amine terminus (or in the case of proline or other N-disubstituted amino acids, R¹ and -AA taken together comprise an N-linked amino acid or peptide linked though such an amino acid).

In certain embodiments, an ascaroside derivative is selected from the group consisting of:

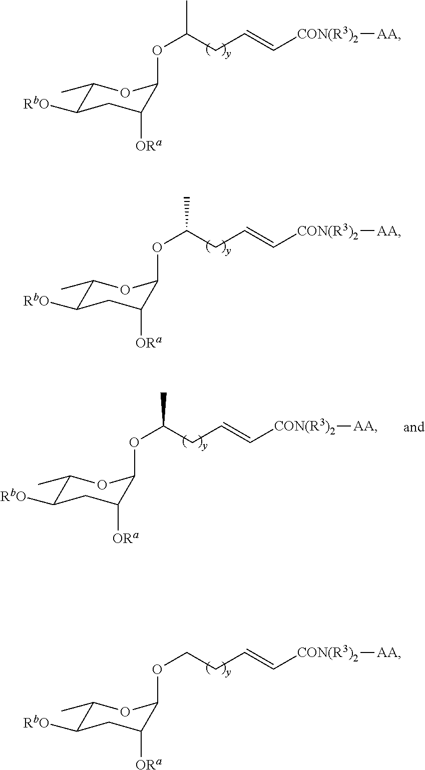

where each of R^{a}, R^{b}, R¹, -AA, and y is as defined above and in the genera and subgenera herein.

In certain embodiments, -AA comprises an alpha amino acid or a peptide linked via an alpha amino acid. In certain embodiments, -AA comprises an L-alpha amino acid or a peptide linked via an L-alpha amino acid. In certain embodiments, -AA is selected from the group of proteinogenic amino acids or a peptide composed of such amino acids. In certain embodiments where -AA is present, R¹ is —H. In certain embodiments, -AA and R¹ taken together comprise N-linked proline, an N-linked proline derivative, or a peptide linked via proline.

In certain embodiments, an ascaroside derivative is selected from the group consisting of:

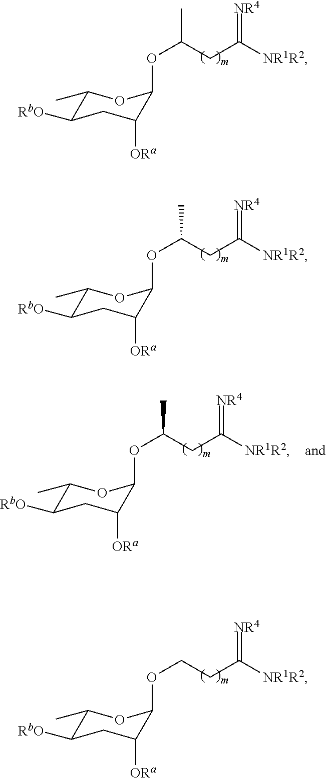

where each of R^{a}, R^{b}, R¹, R², R⁴, and m is as defined above and in the genera and subgenera herein.

In certain embodiments, an ascaroside derivative is selected from the group consisting of:

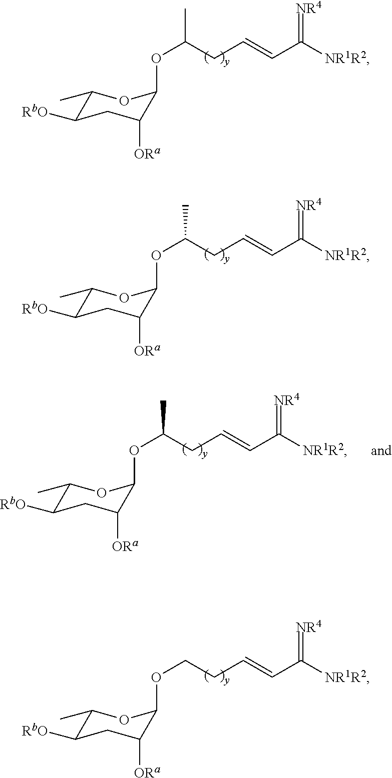

where each of R^{a}, R^{b}, R¹, R², R⁴, and y is as defined above and in the genera and subgenera herein.

In certain embodiments, an ascaroside derivative is selected from the group consisting of:

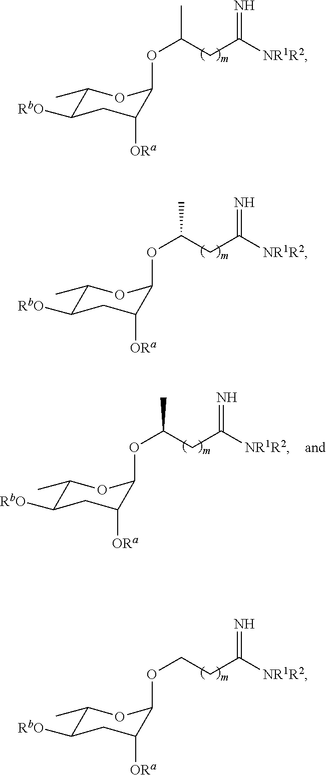

where each of R^{a}, R^{b}, R¹, R², and m is as defined above and in the genera and subgenera herein.

In certain embodiments, an ascaroside derivative is selected from the group consisting of:

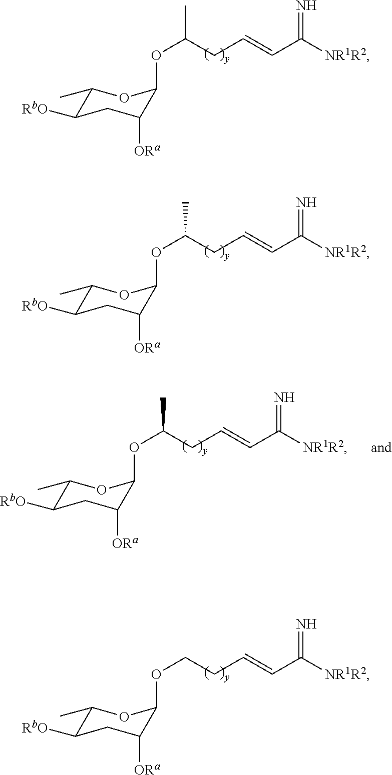

where each of R^{a}, R^{b}, R¹, R², and y is as defined above and in the genera and subgenera herein.

In certain embodiments, an ascaroside derivative is selected from the group consisting of:

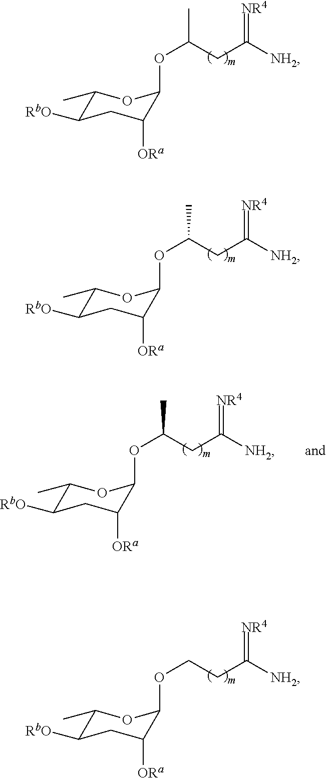

where each of R^{a}, R^{b}, R⁴, and m is as defined above and in the genera and subgenera herein.

In certain embodiments, an ascaroside derivative is selected from the group consisting of:

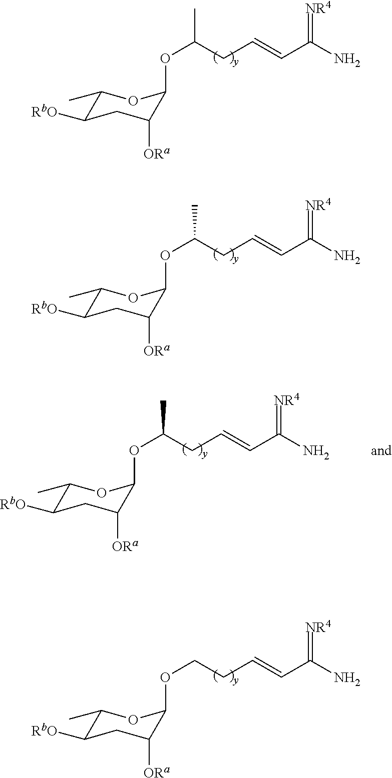

where each of R^{a}, R^{b}, R⁴, and y is as defined above and in the genera and subgenera herein.

In certain embodiments, an ascaroside derivative is selected from the group consisting of:

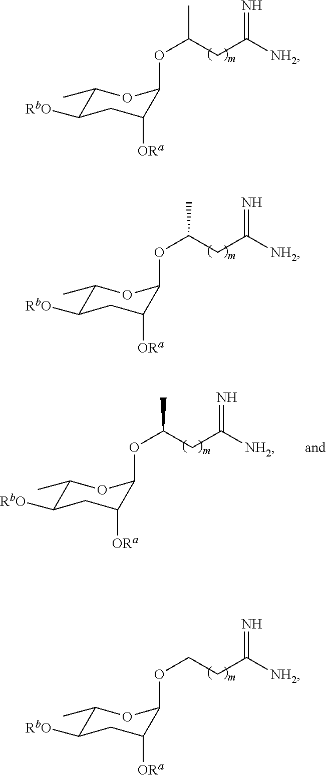

where each of R^{a}, R^{b}, and m is as defined above and in the genera and subgenera herein.

In certain embodiments, an ascaroside derivative is selected from the group consisting of:

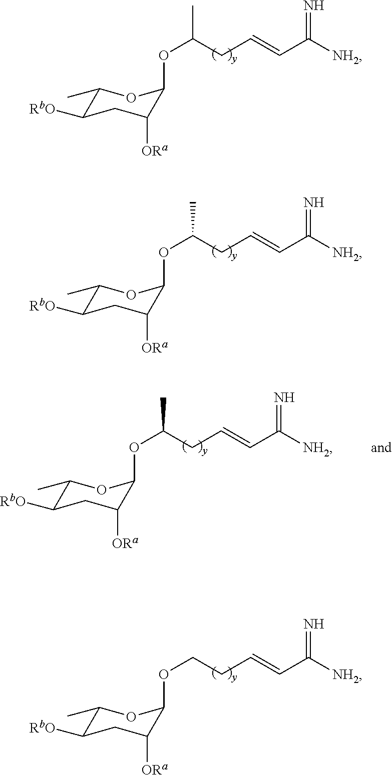

where each of R^{a}, R^{b}, and y is as defined above and in the genera and subgenera herein.

In certain embodiments, an ascaroside derivative is selected from the group consisting of:

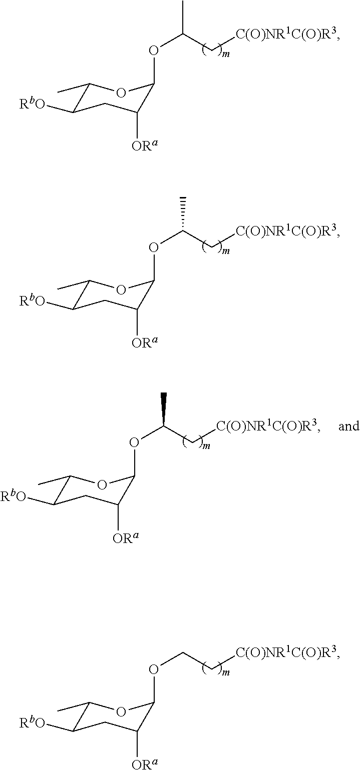

where each of R^{a}, R^{b}, R¹, R³, and m is as defined above and in the genera and subgenera herein.

In certain embodiments, an ascaroside derivative is selected from the group consisting of:

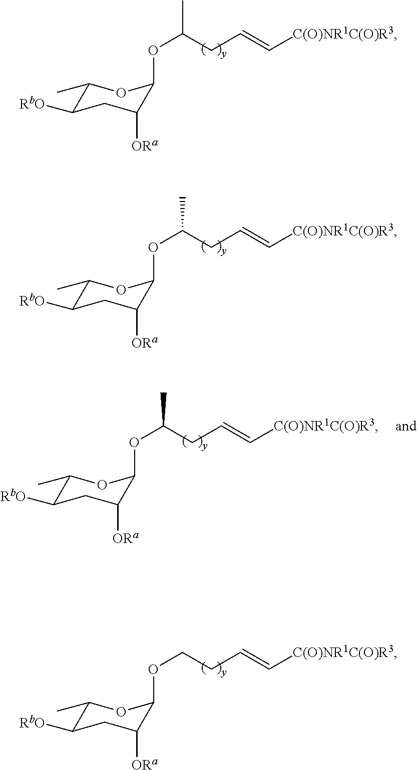

where each of R^{a}, R^{b}, R¹, R³, and y is as defined above and in the genera and subgenera herein.

In certain embodiments, an ascaroside derivative is selected from the group consisting of:

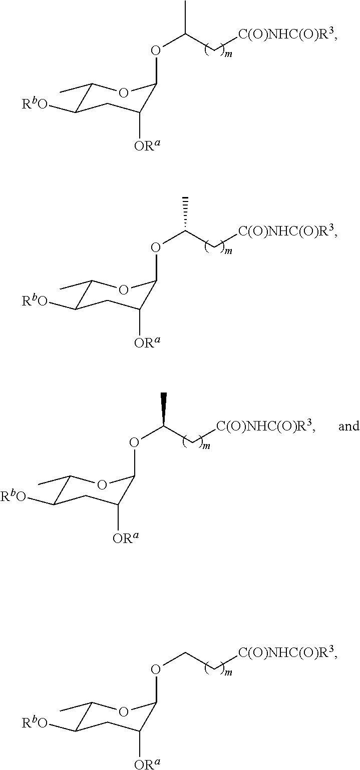

where each of R^{a}, R^{b}, R³, and m is as defined above and in the genera and subgenera herein.

In certain embodiments, an ascaroside derivative is selected from the group consisting of:

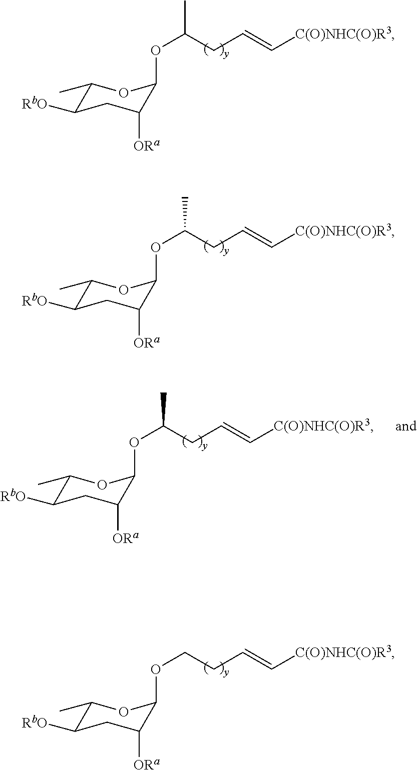

where each of R^{a}, R^{b}, R³, and y is as defined above and in the genera and subgenera herein.

In certain embodiments, an ascaroside derivative is selected from the group consisting of:

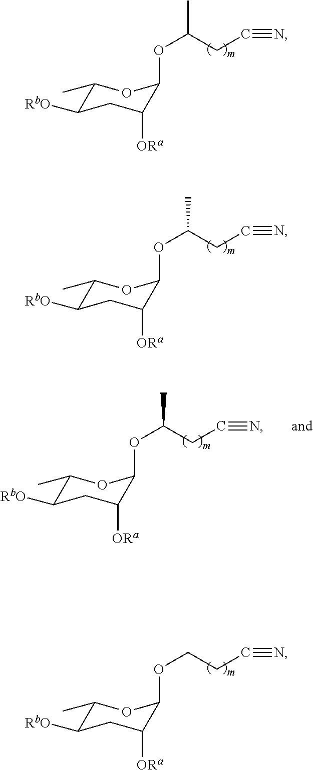

where each of R^{a}, R^{b}, and m is as defined above and in the genera and subgenera herein.

In certain embodiments, an ascaroside derivative is selected from the group consisting of:

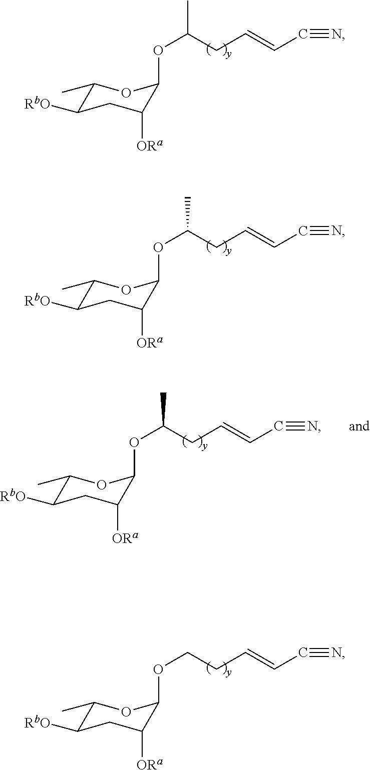

where each of R^{a}, R^{b}, and y is as defined above and in the genera and subgenera herein.

### B. Sulfur-Containing Ascaroside Derivatives

In certain embodiments, —Q is a sulfur-containing functional group. In certain embodiments, —Z comprises an optionally unsaturated, optionally substituted C₂₋₄₀ carbon chain terminating in a sulfur-containing functional group. In certain embodiments, —Z comprises an optionally unsaturated, optionally substituted C₂₋₄₀ carbon chain terminating in a functional group selected from the group consisting of: thioacid, thioester, dithioester, thioamide, and thioimide. In certain embodiments, —Z terminates in a thioacid functional group. In certain embodiments, —Z terminates in a thioester functional group. In certain embodiments, —Z terminates in a dithioester functional group. In certain embodiments, —Z terminates in a thioamide functional group. In certain embodiments, —Z terminates in a thioimide functional group.

In certain embodiments, —Q is selected from the group consisting of: —C(S)OR¹, —C(O)—Sₓ—SR¹, —C(S)—Sₓ—SR¹, —C(S)NR¹R², —C(S)NR¹C(O)R³, —C(O)NR¹C(S)R³, and —C(S)NR¹C(S)R³; where x is an integer between 0 and 6, and each of R¹, R², and R³ is as defined above and in the genera and subgenera herein.

In certain embodiments, —Q is a sulfur-containing functional group and at least one of R¹ and R² is —H. In certain embodiments, —Q is a sulfur-containing functional group and both R³ groups are —H. In certain embodiments, —Q is a sulfur-containing functional group and at least one of R¹ and R² is an optionally substituted C₁₋₂₀ aliphatic group. In certain embodiments, —Q is a sulfur-containing functional group and both R¹ and R² are an optionally substituted C₁₋₂₀ aliphatic group which may be the same or different. In certain embodiments, —Q is a sulfur-containing functional group and at least one of R¹ and R² is an optionally substituted C₁₋₁₂ aliphatic group. In certain embodiments, —Q is a sulfur-containing functional group and at least one of R¹ and R² is an optionally substituted C₁₋₈ aliphatic group. In certain embodiments, —Q is a sulfur-containing functional group and at least one of R¹ and R² is an optionally substituted C₁₋₆ aliphatic group. In certain embodiments, —Q is a sulfur-containing functional group and at least one of R¹ and R² is selected from methyl, ethyl, n-propyl, i-propyl, n-butyl, sec-butyl, and t-butyl. In certain embodiments, —Q is a sulfur-containing functional group and at least one of R¹ and R² is —CH₂CH₂OH. In certain embodiments, —Q is a sulfur-containing functional group and at least one of R¹ and R² is —CH₂CH₂OR³, where R³ is as defined above and in the genera and subgenera herein. In certain embodiments, —Q is a sulfur-containing functional group and at least one of R¹ and R² is an optionally substituted aromatic group. In certain embodiments, —Q is a sulfur-containing functional group and at least one of R¹ and R² comprises a glycoside. In certain embodiments, —Q is a sulfur-containing functional group and at least one of R¹ and R² comprises an amino acid. In certain embodiments, —Q is a sulfur-containing functional group and at least one of R¹ and R² comprises a peptide. In certain embodiments, —Q is a sulfur-containing functional group and at least one of R¹ and R² comprises a nucleotide.

In certain embodiments, an ascaroside derivative is selected from the group consisting of:

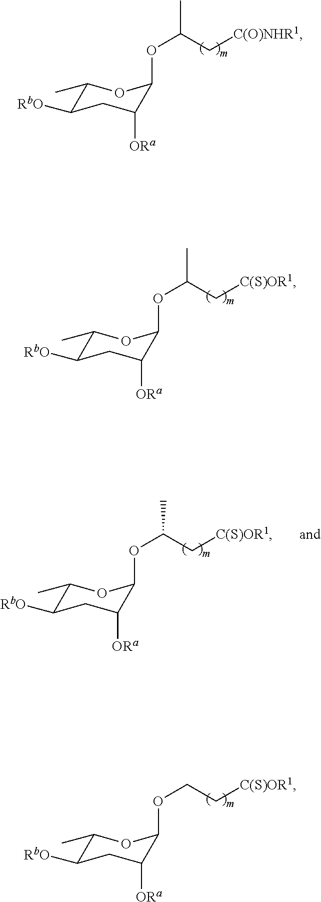

where each of R^{a}, R^{b}, R¹, and m is as defined above and in the genera and subgenera herein.

In certain embodiments, an ascaroside derivative is selected from the group consisting of:

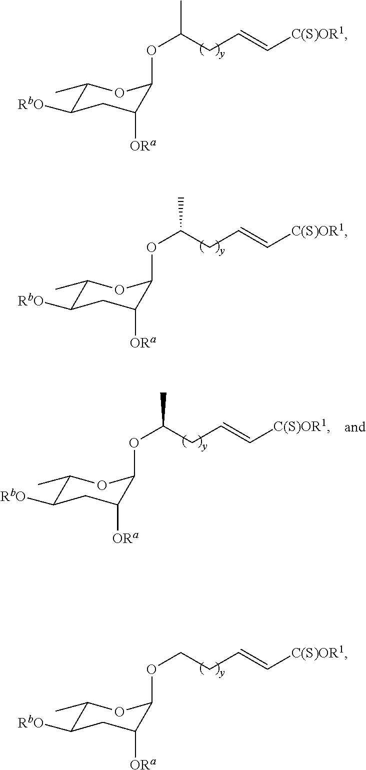

where each of R^{a}, R^{b}, R¹, and y is as defined above and in the genera and subgenera herein.

In certain embodiments, an ascaroside derivative is selected from the group consisting of:

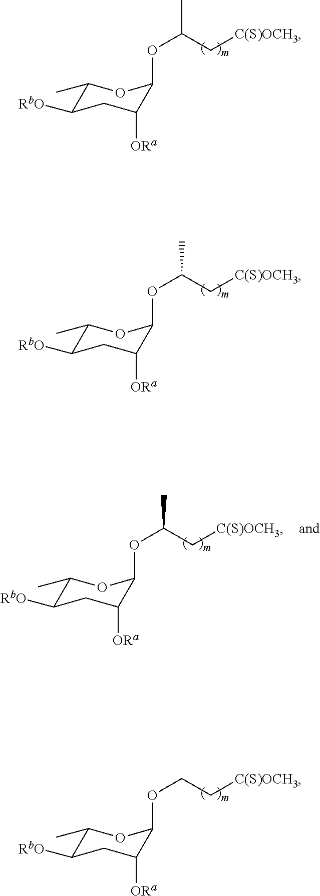

where each of R^{a}, R^{b}, R¹, and m is as defined above and in the genera and subgenera herein.

In certain embodiments, an ascaroside derivative is selected from the group consisting of:

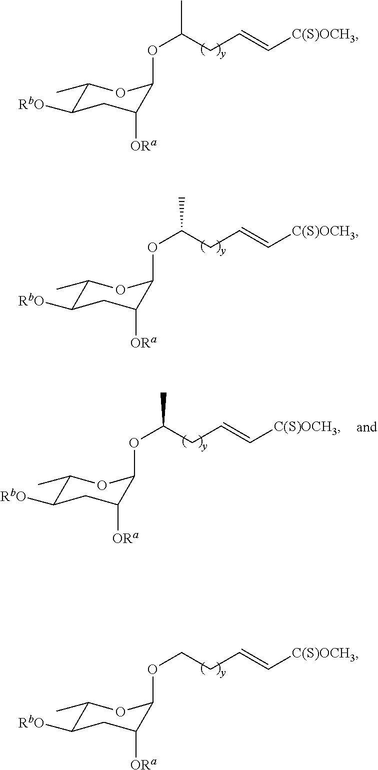

where each of R^{a}, R^{b}, R¹, and y is as defined above and in the genera and subgenera herein.

In certain embodiments, an ascaroside derivative is selected from the group consisting of:

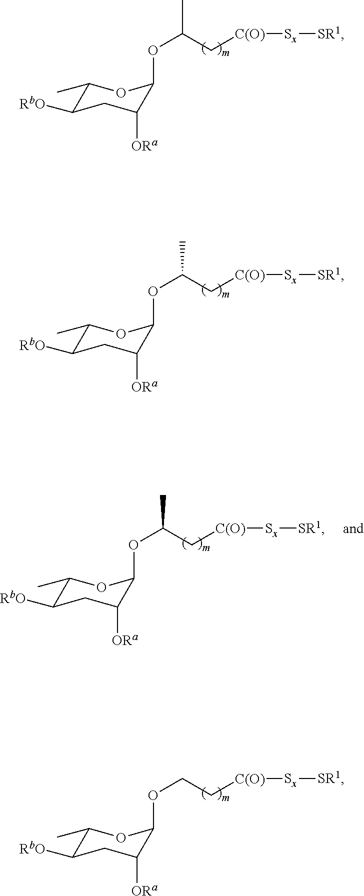

where each of R^{a}, R^{b}, R¹, X, and m is as defined above and in the genera and subgenera herein.

In certain embodiments, an ascaroside derivative is selected from the group consisting of:

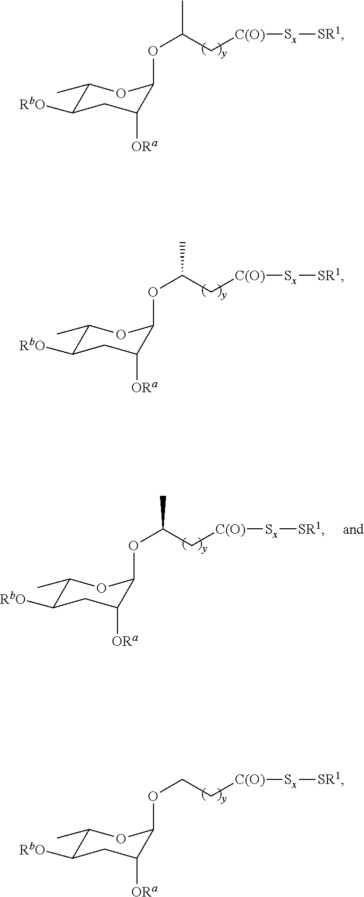

where each of R^{a}, R^{b}, R¹, X, and y is as defined above and in the genera and subgenera herein.

In certain embodiments, an ascaroside derivative is selected from the group consisting of:

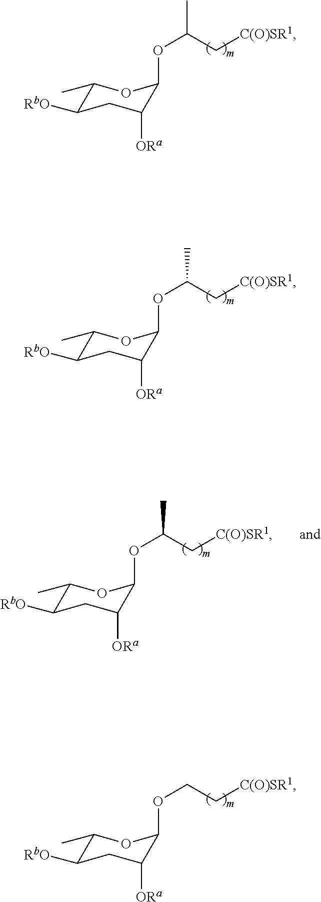

where each of R^{a}, R^{b}, R¹, and m is as defined above and in the genera and subgenera herein.

In certain embodiments, an ascaroside derivative is selected from the group consisting of:

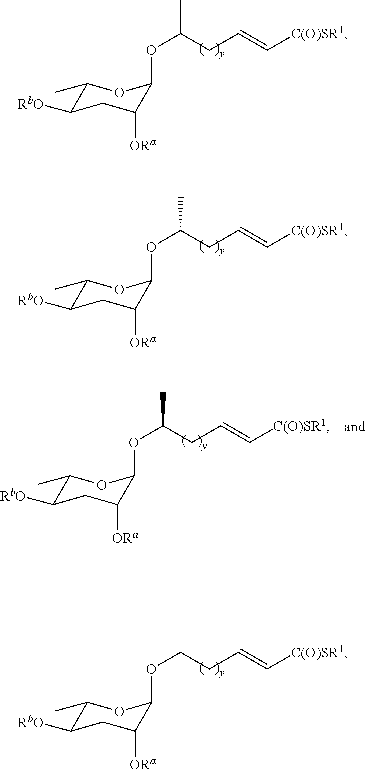

where each of R^{a}, R^{b}, R¹, and y is as defined above and in the genera and subgenera herein.

In certain embodiments, an ascaroside derivative is selected from the group consisting of:

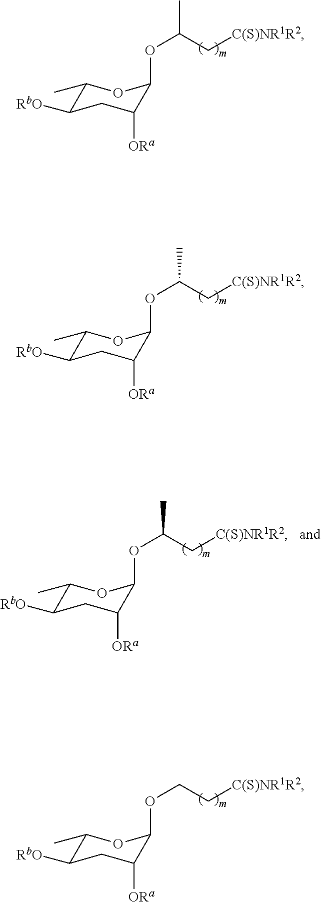

where each of R^{a}, R^{b}, R¹, R², and m is as defined above and in the genera and subgenera herein.

In certain embodiments, an ascaroside derivative is selected from the group consisting of:

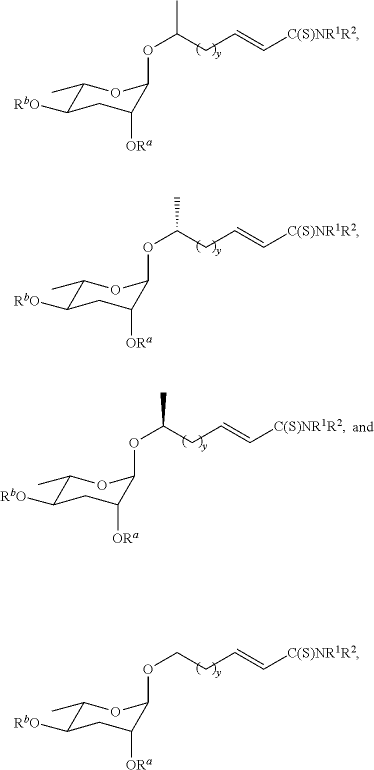

where each of R^{a}, R^{b}, R¹, R², and y is as defined above and in the genera and subgenera herein.

In certain embodiments, an ascaroside derivative is selected from the group consisting of:

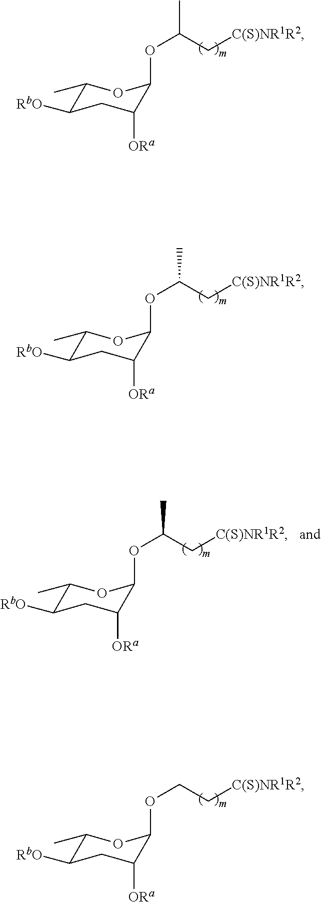

where each of R^{a}, R^{b}, R¹, R², and m is as defined above and in the genera and subgenera herein.

In certain embodiments, an ascaroside derivative is selected from the group consisting of:

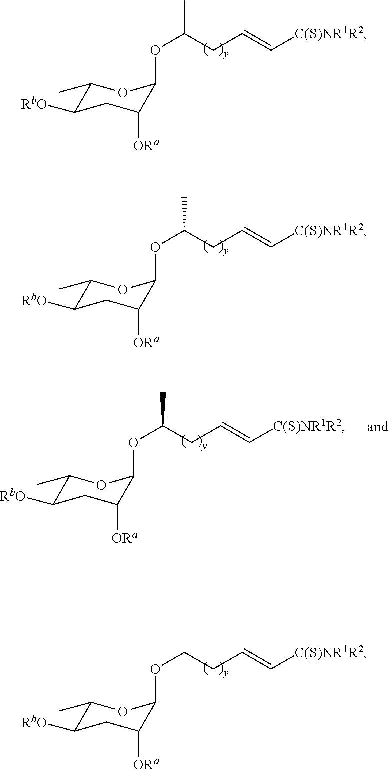

where each of R^{a}, R^{b}, R¹, R², and y is as defined above and in the genera and subgenera herein.

### C. Oligomeric Ascaroside Derivatives

As noted above, in certain embodiments, provided ascaroside derivatives and compositions may comprise dimers or oligomers containing more than one ascaroside moiety. Such compounds may arise for example where one or both of the groups R^{a} or R^{b} comprise a linkage to another ascaroside molecule. Such dimers or oligomers may be symmetrical (i.e. where there is an axis of symmetry between two halves of a dimer) or asymmetrical (e.g. where two ascarosides are linked through different positions). Furthermore dimeric or oligomeric structures may be substantially pure compounds with a defined structure, or they may comprise a mixture of numerous components (e.g., a statistical mixture of oligomers with differing numbers of connected monomeric units and/or containing two or more differing isomers or linker structures connecting the monomeric units.

In other embodiments where provided ascaroside derivatives comprise dimers or oligomers, these may arise via linkages through the sidechains and may comprise linkages involving the nitrogen- or sulfur-containing functional groups. Such compounds may arise for example where one or both the groups R¹, R², R³, or R⁴ comprise a linkage to another ascaroside molecule. By way of illustration, exemplary non-limiting dimers are shown below:

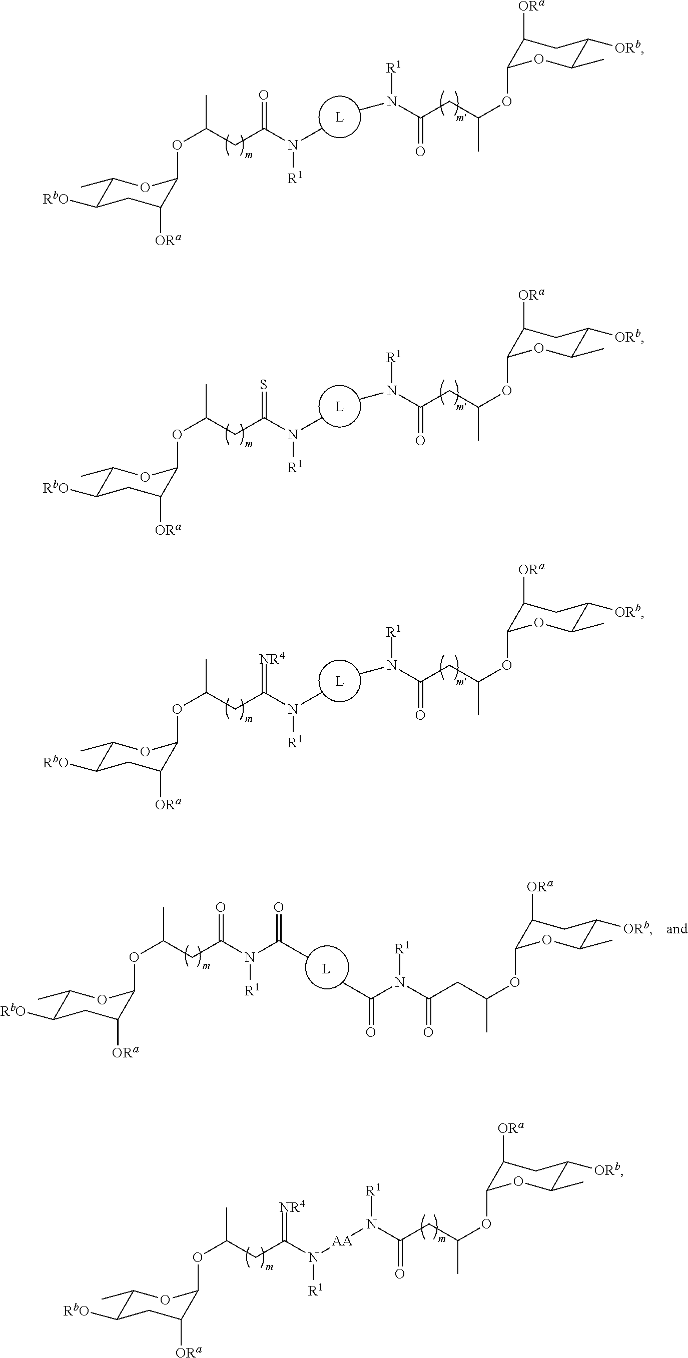

where
each of R¹, R⁴, R^{a}, R^{b}, AA, and m are as defined above and in the genera and subgenera herein where these variables may be the same or different at each occurrence in a dimer or oligomeric structure, and the moiety \textcircled{L} comprises an optionally substituted aliphatic, heteroaliphatic aromatic, or heteroaromatic group, or a polymer.

It will be appreciated that other variations similar to the dimers depicted above are also possible. For example dimers linked through other nitrogen- and sulfur-containing functional groups defined herein are possible as are heterodimeric analogs where the two ascarosides are linked via different functional groups. Similarly, oligomeric analogs of similar structure are possible when the linker moiety \textcircled{L} has the capacity to form three or more covalent linkages to ascarosides. These variations and their combinations are contemplated and encompassed within the provided ascaroside derivatives and the compositions containing them.

### D. Other Substituents and Characteristics of Ascaroside Derivatives

As described above, the ascarylose sugar in the provided ascaroside derivatives can be substituted or unsubstituted (i.e. there can be functional groups other than —OH at 2- and 4-positions of the sugar or, stated differently, variables R^{a} and/or R^{b} can be other than —H in any of the formulae herein). In certain embodiments, in any of the formulae above, R^{a} is —H. In certain embodiments, in any of the formulae above, R^{b} is —H. In certain embodiments, R^{a} and R^{b} are the same. In certain embodiments R^{a} and R^{b} are both —H. In certain embodiments, R^{a} and R^{b} are different. In certain embodiments, R^{a} is —H, and R^{b} is other than —H. In certain embodiments, R^{a} is other than —H and R^{b} is —H. In certain embodiments, R^{a} is —H and R^{b} is p-hydroxybenzoate. In certain embodiments, R^{a} is —H and R^{b} is indole-3-carboxylate. In certain embodiments, R^{a} is —H and R^{b} is (E)-2-methyl-2-butenoate. In certain embodiments, R^{a} is —H and R^{b} is picolinate. In certain embodiments, R^{a} is —H and R^{b} is nicotinate. In certain embodiments, R^{a} is —H and R^{b} is (R)-2-hydroxy-2-(4-hydroxyphenyl)ethyl)amino)-4-oxobutanoate. In certain embodiments, R^{a} is —H and R^{b} is 4-((4-hydroxyphenethyl)amino)-4-oxobutanoate. In certain embodiments, R^{a} comprises a glycoside, amino acid, a peptide, or nucleotide. In certain embodiments, R^{b} comprises a glycoside, amino acid, a peptide, or nucleotide. In certain embodiments, R^{a} comprises a linkage to a second ascaroside molecule. In certain embodiments, R^{b} comprises a linkage to a second ascaroside molecule. In certain embodiments, R^{a} comprises a sugar. In certain embodiments, R^{b} comprises a sugar.

In certain embodiments R^{a} and R^{b} are both —H, and —Z has a structure selected from the group consisting of: —CH(CH₃)(CH₂)ₙ—Q, —CH(CH₃)(CH₂)ₙCH═CH—Q, —CH(CH₃)(CH₂)ₙ—CH═CH—(CH₂)ₙ—Q, —CH(CH₃)—(CH₂)ₙ—CH(OH)—CH₂—Q, —CH(CH₃)—(CH₂)ₙ—CH(OH)—Q, —(CH₂)ₙ—Q, —(CH₂)ₙ—CH═CH—Q, —(CH₂)ₙ—CH═CH—(CH₂)ₙ—Q, —CH(CH₃)(CH₂)ₙ—CH═CH—(CH₂)ₙ—Q, —(CH₂)ₙ—CH(OH)—CH₂—Q, and —(CH₂)ₙ—CH(OH)—Q, where n is independently at each occurrence an integer between 1 and 24 and —Q is as defined above and in the genera and subgenera herein.

In certain embodiments, the variable m in any of the structures herein is an integer from 1 to 24 (inclusive). In certain embodiments m is an integer between 1 and 4 inclusive. In certain embodiments m is an integer between 4 and 8 inclusive. In certain embodiments m is an integer between 5 and 11 inclusive. In certain embodiments m is an integer between 6 and 10 inclusive. In certain embodiments m is an integer between 7 and 9 inclusive. In certain embodiments m is 7. In certain embodiments m is 8. In certain embodiments m is 9. In certain embodiments m is an integer greater than 6. In certain embodiments m is an integer less than 20.

In certain embodiments the variable y in any of the structures herein is an integer from 1 to 20 (inclusive). In certain embodiments y is an integer between 1 and 8 inclusive. In certain embodiments y is an integer between 1 and 6 inclusive. In certain embodiments y is an integer between 2 and 4 inclusive. In certain embodiments vis an integer between 4 and 12 inclusive. In certain embodiments y is an integer between 7 and 11 inclusive. In certain embodiments y is an integer between 8 and 10 inclusive. In certain embodiments y is 2. In certain embodiments vis 3. In certain embodiments y is 4. In certain embodiments y is an integer greater than 2. In certain embodiments y is an integer less than 10.

In certain embodiments the variable n in any of the structures herein is an integer from 1 to 20 (inclusive). In certain embodiments n is an integer between 1 and 8 inclusive. In certain embodiments n is an integer between 1 and 6 inclusive. In certain embodiments n is an integer between 2 and 4 inclusive. In certain embodiments n is an integer between 4 and 12 inclusive. In certain embodiments n is an integer between 7 and 11 inclusive. In certain embodiments n is an integer between 8 and 10 inclusive. In certain embodiments n is 2. In certain embodiments n is 3. In certain embodiments n is 4. In certain embodiments n is an integer greater than 2. In certain embodiments n is an integer less than 10.

In certain embodiments, ascaroside derivatives of the present invention have utility as precursors to ascarosides having a carboxylic acid moiety at the end of the sidechain. As shown in the scheme below, a provided ascaroside having a sidechain terminating a —Q group (as defined herein) may produce a corresponding ascaroside carboxylic acid when hydrolyzed or metabolized.

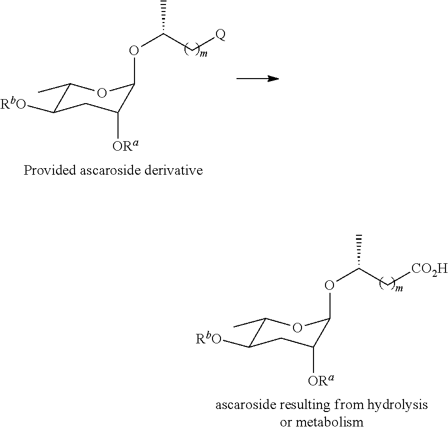

This scheme allows for a situation where the identity of any of m, R^{a} and R^{b} in the product may be the same or different from the identity of those variables in the provided ascaroside derivative. The situations where any one or more of m, R^{a} and R^{b} are different in the provided ascaroside derivative versus the resulting hydrolysis or metabolism product are specifically encompassed within certain embodiments of the invention. For example, this situation may arise through concomitant hydrolysis of the functional group —Q and hydrolysis of R^{a} or R^{b}, (or a functional group composing R^{a} or R^{b}). Another example would be where the provided ascaroside derivative undergoes shortening of the sidechain (i.e. through beta oxidation) resulting in the value of m in the product being different (smaller) than the value of m in the provided ascaroside derivative. In the latter case, such chain shortening may be part of the mechanism by which the moiety —Q is metabolized, or it may be a consequence of an additional process that occurs either subsequent to or concomitantly with hydrolysis or metabolism of the functional group —Q.

In certain embodiments, provided ascaroside derivatives are characterized in that the moiety —Q undergoes hydrolysis or metabolism to provide an ascaroside acid (or an ester or salt thereof) having a sidechain that is otherwise identical to the sidechain of the provided ascaroside derivative (i.e. where the sidechain of the provided ascaroside derivative and the resulting metabolite or hydrolysate have the same carbon skeleton and differ only in the identity of the terminal functional group). For example, the provided amidine derivative shown below hydrolyzes to ascr #18, and here the two structures are identical except for the functional group at the end of the sidechain.

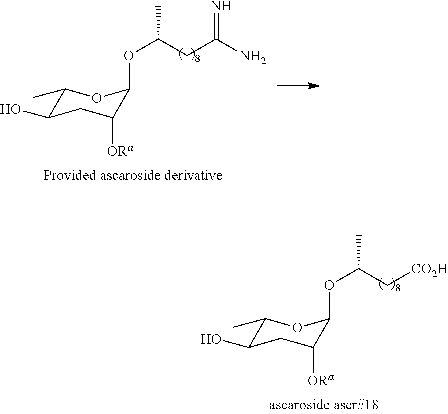

In certain embodiments, R^{a} and R^{b} in the provided ascaroside derivative are unchanged before and after hydrolysis or metabolism of the derivative (i.e. each of R^{a} and R^{b} is the same in the provided ascaroside derivative and in the ascaroside resulting from hydrolysis or metabolism of the derivative). In certain embodiments, R^{a} and/or R^{b} in the provided ascaroside derivative is different after hydrolysis or metabolism of the derivative. In certain embodiments, R^{a} and/or R^{b} in the provided ascaroside derivative is other than —H and becomes —H upon hydrolysis or metabolism of the ascaroside derivative. In certain embodiments, R^{a} and/or R^{b} in the provided ascaroside derivative is —H and becomes other than —H upon hydrolysis or metabolism of the ascaroside derivative. In certain embodiments, both R^{a} and R^{b} are —H in the provided ascaroside derivative and in the resulting hydrolysis or metabolism product.

Specific ascaroside derivatives that are useful for the present invention include, but are not limited to molecules that, upon hydrolysis or metabolism, liberate the ascarosides ascr #7 or ascr #18 (or esters or salts thereof).

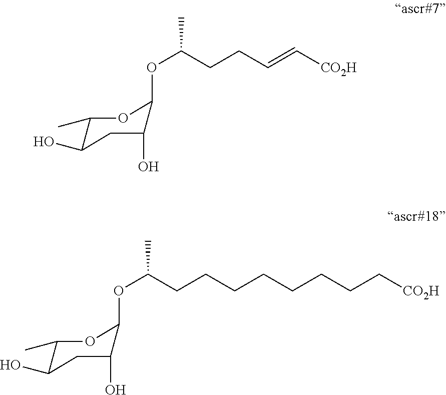 [SMILES: CC1OC(O[C@H](C)CC/C=C/C(=O)O)C(O)CC1O ; CC1OC(O[C@H](C)CCCCCCCCC(=O)O)C(O)CC1O]

In certain embodiments, a provided ascaroside derivative is one which, upon hydrolysis or metabolism, liberates an ascaroside selected from the group consisting of: ascr #9, ascr #12, ascr #14, ascr #1, ascr #10, ascr #16, ascr #18, ascr #20, ascr #22, ascr #24, ascr #26, ascr #28, ascr #30, ascr #32, ascr #34, and ascr #36. In certain embodiments, a provided ascaroside derivative is one which, upon hydrolysis or metabolism, liberates an ascaroside selected from the group consisting of: ascr #10, ascr #16, ascr #18, ascr #20, ascr #22, and ascr #24. In certain embodiments, a provided ascaroside derivative is one which, upon hydrolysis or metabolism, liberates an ascaroside selected from the group consisting of: ascr #9, ascr #14, ascr #10, and ascr #18. In certain embodiments, such derivatives have a sidechain with the same structure as the liberated ascaroside aside from the structure of the terminal moiety.

In certain embodiments, an ascaroside derivative is one which, upon hydrolysis or metabolism, liberates an ascaroside selected from the group consisting of: ascr #5, oscr #9, oscr #12, oscr #1, oscr #14, oscr #10, oscr #16, oscr #18, oscr #20, oscr #22, oscr #24, oscr #26, oscr #28, oscr #30, oscr #32, oscr #34, and oscr #36. In certain embodiments, a provided ascaroside derivative is one which, upon hydrolysis or metabolism, liberates an ascaroside selected from the group consisting of: oscr #10, oscr #16, oscr #18, oscr #20, and oscr #22. In certain embodiments, a provided ascaroside derivative is one which, upon hydrolysis or metabolism, liberates an ascaroside selected from the group consisting of: bhas #5, oscr #9, oscr #12, oscr #1, oscr #14, oscr #10, oscr #16, oscr #18, oscr #20, oscr #22, oscr #24, oscr #26, oscr #28, oscr #30, oscr #32, oscr #34, and oscr #36. In certain embodiments, a provided ascaroside derivative is one which, upon hydrolysis or metabolism, liberates an ascaroside selected from the group consisting of: oscr #10, oscr #16, oscr #18, oscr #20, and oscr #22.

In certain embodiments, a provided ascaroside derivative is one which, upon hydrolysis or metabolism, liberates an ascaroside selected from the group consisting of: bhas #9, bhas #10, bhas #16, bhas #18, bhas #22, bhas #24, bhas #26, bhas #28, bhas #30, bhas #32, bhas #34, bhas #36, bhas #38, bhas #40, and bhas #42.

In certain embodiments, a provided ascaroside derivative is one which, upon hydrolysis or metabolism, liberates an ascaroside selected from the group consisting of: bhos #10, bhos #16, bhos #18, bhos #22, bhos #24, bhos #26, bhos #28, bhos #30), bhos #32, bhos #34, bhos #36, bhos #38, bhos #40, and bhos #42.

In certain embodiments, a provided ascaroside derivative is one which, upon hydrolysis or metabolism, liberates an ascaroside selected from the group consisting of: ascr #18, oscr #16, oscr #17, oscr #15, bhas #18, bhos #16, glas #18, dhas #18, ibha #18, ibho #16, icas #18, icos #15, icos #16, and any combination of two or more of these.

In certain embodiments, provided ascaroside derivatives are characterized in that their uptake by an organism contacted with the derivative is enhanced relative to the uptake of the corresponding ascaroside carboxylic acid or ester under the same conditions. In certain embodiments, provided ascaroside derivatives are characterized in that their uptake by an organism contacted with the derivative is faster than uptake of the corresponding ascaroside carboxylic acid or ester under the same conditions. In certain embodiments, provided ascaroside derivatives are characterized in that their uptake by an organism contacted with the derivative is more complete than uptake of the corresponding ascaroside carboxylic acid or ester under the same conditions (e.g. a higher percentage of the material in contact with the organism is taken up).

In certain embodiments, a provided ascaroside derivatives is characterized in that it is at least partially converted to the corresponding ascaroside carboxylic acid or ester after uptake by an organism (e.g. the nitrogen- or sulfur-containing functional group on the sidechain of the ascaroside derivative is hydrolyzed or metabolized after uptake resulting in an ascaroside with a carboxylic acid or ester group on its sidechain). In certain embodiments, the provided ascaroside derivative is characterized in that it is at least partially hydrolyzed to the corresponding ascaroside carboxylic acid after uptake by an organism. In certain embodiments, substantially all of the provided ascaroside derivative is converted to the corresponding ascaroside carboxylic acid or ester after uptake by an organism. In certain embodiments, the provided ascaroside derivative is at least partially hydrolyzed to the corresponding ascaroside carboxylic acid prior to uptake by an organism. In certain embodiments, substantially all of the provided ascaroside derivative is hydrolyzed to the corresponding ascaroside carboxylic acid prior to uptake by an organism.

In certain embodiments, provided ascaroside derivatives are characterized in that the extent or rate of metabolism of the derivative by an organism contacted or treated with the derivative is reduced relative to the metabolism of the corresponding ascaroside carboxylic acid or ester under the same conditions. In certain embodiments, provided ascaroside derivatives are characterized in that their metabolism by an organism contacted with the derivative is slower than metabolism of the corresponding ascaroside carboxylic acid or ester under the same conditions. In certain embodiments, provided ascaroside derivatives are characterized in that the extent of their metabolism by an organism contacted with the derivative is less than that of the corresponding ascaroside carboxylic acid or ester under the same conditions (e.g. a smaller percentage of the material in contact with the organism is metabolized).

In certain embodiments, provided ascaroside derivatives are characterized in that they are at least partially converted after application (e.g. after applying to surface of a plant or organism or the immediate environment of such a plant or organism) to the corresponding ascaroside carboxylic acid or ester. In some embodiments, use of an ascaroside derivative can provide various formulation benefits as compared with the corresponding ascaroside (e.g., improved handling characteristics and/or simplified or improved incorporation within agrichemical or pharmaceutical compositions). In some embodiments, use of a provided ascaroside derivative can also provide additional benefits to plants to which the ascaroside derivative is applied, beyond the benefits generally afforded by application of an ascaroside, e.g., by providing other beneficial molecules such as nutrients, hormones, peptides or amino acids that are liberated when the nitrogen- or sulfur-containing functional group is hydrolyzed.

Ascarosides can be obtained from natural sources (e.g., nematodes) or they may be prepared synthetically. Ascarosides and their derivatives can also be prepared synthetically, for example, by converting 1-O-substituted rhamnose to 1-O-substituted ascarylose. An exemplary method of preparing ascarosides includes: providing as a feedstock a 1-O-substituted rhamnose; forming a mono-sulfonate ester at the 3-OH group of the feedstock; and treating the mono-sulfonate ester with a hydride source to form a 1-O substituted ascarylose. In certain embodiments, forming the mono-sulfonate ester is conducted on a substrate without hydroxyl protecting groups at the 2- or 4-position of the rhamnose feedstock. In certain embodiments, such methods comprise contacting the feedstock with a sulfonating agent (i.e., a sulfonyl halide, sulfonic anhydride or similar reagent) in the presence of a Lewis acid. Specific details regarding the synthesis of 1-O-substituted ascarylose can be found in PCT Application No. PCT/IB2021/056981 (WO2022/024067), which is incorporated herein by reference.

In certain embodiments, ascaroside derivatives of the present invention have utility as bioactive molecules (e.g. molecules having biological activity in there own right without the need for metabolism or conversion to another structure). In certain embodiments, provided ascaroside derivatives have utility in agricultural applications. In certain embodiments, provided ascaroside derivatives have utility as modulators of plant defenses, plant growth regulators, pesticides, fungicides, antimicrobial agents, biostimulants, antivirals, antibacterial agents, antihelminthic agents, pheromones, and the like. In certain embodiments, the present invention provides methods of treating a plant with any one of the provided ascaroside derivatives to improve the health of the plant.

In certain embodiments, provided ascaroside derivatives have utility in pharmaceutical, nutritional or therapeutic treatments for mammals or other animals. In certain embodiments, provided ascaroside derivatives have utility in the treatment or amelioration of human diseases or health disorders. In certain embodiments, provided ascaroside derivatives have utility as immunomodulators, antibiotics, antiproliferative agents, antihypertensive agents, antiviral agents, and other pharmaceutical uses. In certain embodiments, the present invention provides methods of treating an animal with any one of the provided ascaroside derivatives to improve the health or wellbeing of the animal.

Ascaroside derivatives described herein can be synthesized from an isolated or synthesized ascaroside using known reactions for transformations of functional groups (e.g. such as contacting an ascaroside with an amine under dehydrating conditions convert a carboxylic acid to a corresponding amide). In other embodiments, the ascaroside derivatives may be synthesized from starting materials other than ascarosides—for example, a sidechain already functionalized with a nitrogen- or sulfur-containing functional group (or a precursor to such a functional group) can be appended to ascarylose or a suitable ascarylose precursor. An example of the latter would include coupling 10-hydroxydecanitrile to an ascarylose substrate or precursor to directly access an ascaroside derivative containing a nitrile group on the sidechain.

The ascaroside derivatives disclosed herein can, in some embodiments, exhibit enhanced physical properties as compared with a corresponding ascaroside molecule having a carboxylic acid instead of the nitrogen- or sulfur-containing functional group on the sidechain. For example, in some embodiments, provided ascaroside derivatives can exhibit greater solubility in certain solvents (e.g., water or organic solvents) than the corresponding ascaroside with a carboxylic acid. This can simplify the preparation of certain formulations. In some embodiments, providing an ascaroside derivative (rather than as its free acid) renders it more compatible with certain components of a desired composition thus stabilizing the composition. In some embodiments, e.g., where —Q comprises a functional group or structure that can act as a plant nutrient or hormone the ascaroside derivatives herein can provide benefits beyond formulation benefits, e.g., providing nutrients or hormones to a plant to which (or near which) it is applied. In some embodiments, the ascaroside derivatives described herein have improved application characteristics (e.g. enhanced ability to wet or adhere to surfaces such as leaves or skin), enhanced bioavailability, or enhanced uptake (e.g. by a plant, microorganism, or mammal).

In certain embodiments, the ascaroside derivatives provided herein comprise “multifunctional” ascaroside derivatives. In certain embodiments such multifunctional ascaroside derivatives combine ascarosides with molecules having other different, or complementary biological activity. In certain embodiments, such multifunctional products comprise an ascaroside covalently linked to a second molecule having independent bioactivity as a pesticide (e.g. insecticides, fungicides, herbicides, miticides, nematicides), as an antimicrobial agent (e.g. antibiotics, antifungals, etc), as antiviral agents, as signaling molecules, (e.g. pheromones, hormones, etc.) or as vitamins or nutrients. Such multifunctional products can avoid the need for multiple product applications (for example of a drug or an agricultural treatment) saving costs and/or simplifying methods of treatment. In certain embodiments, the second bioactive molecule is covalently linked to the ascaroside through a nitrogen- or sulfur-containing functional group such as those described herein. In certain embodiments, the linkage between the second bioactive molecule and the ascaroside is hydrolytically or metabolically labile, such that the second bioactive molecule is liberated from the ascaroside upon hydrolysis or metabolism of the multifunctional ascaroside derivative.

### II. METHODS OF USE

In certain embodiments, the ascaroside derivatives described herein and compositions containing such derivatives are useful for improving the pathogen resistance, health, vigor, or agricultural yield of plants. Therefore, in one aspect, the invention provides methods of treating plants. In certain embodiments, such methods comprise a step of contacting a plant, a seed, the soil surrounding a plant, or the soil in which seeds/seedlings are to be planted, or a solution in which the plant is in contact (e.g. in a hydroponic system) with a composition containing any one or more of the ascaroside derivatives described herein.

In some embodiments, the ascaroside derivative is applied to a portion of a plant, e.g., one or more of a root, stem, bark, leaf, seed, and/or flower. Such methods can be conducted at any one or more stages in the life cycle of a plant, e.g., from seed to seedling to growing plant to just prior to or after harvest.

The disclosed treatment methods can, in some embodiments, protect growing plants in the manner described in U.S. Pat. No. 10,136,595, which is incorporated by reference herein in its entirety. For example, such methods can enhance pathogen resistance and/or induce or prime one or more plant defense responses (thereby inhibiting pathogen growth and/or infestation) in a plant to (or near) which the ascaroside derivative is applied. Pathogens against which the disclosed methods can enhance resistance include, but are not limited to, fungi, oomycetes, bacteria, nematodes, viruses, and insects, e.g., including but not limited to, Pseudomonas syringae, Phytophthora infestans, Blumeria graminis, Heterodera schachtii, Meloidogyne incognita, Meloidogyne hapla, and turnip crinkle virus.

The disclosed treatment methods can, in some embodiments, protect growing plants in the manner described in U.S. Patent Application Publication No. US2022/0183291, which is incorporated by reference herein in its entirety. For example, such methods can repel nematodes from a plant, or can be taken up by a plant and metabolized to compounds that repel nematodes from the treated plant.

The exact method by which a plant or soil is treated with an ascaroside derivative is not particularly limited. Treatment of plants and/or soil according to the present disclosure can be carried out, e.g., by immersion, spraying, evaporation, fogging, scattering, painting on, side dressing, or in-furrow application. For example, in certain embodiments, plants or soil can be sprayed with a suitable liquid composition, a solid plastic mulch composition can be applied on soil around plants, and/or a granular composition can be provided for in-furrow application or side-dressing.

The type of plants that can be treated according to the presently disclosed methods is not particularly limited and can be, for example, fruit and vegetable plants, turfgrass, trees, and shrubs. Non-limiting examples of plants that can be treated according to the disclosed methods include, but are not limited to, plants selected from the group consisting of tobacco, Arabidopsis, tomato, barley, potato, sweet potato, yam, cotton, soybean, strawberry, sugar beet, corn, rice, wheat, rye, oat, sorghum, millet, bean, pea, apple, banana, pear, cherry, peach, plum, apricot, almond, grape, kiwi, mango, melon, papaya, walnut, hazelnut, pistachio, raspberry, blackberry, loganberry, blueberry, cranberry, orange, lemon, grapefruit, tangerine, lettuce, carrots, onions, broccoli, cabbage, avocado, cocoa, cassava, cotton, and flax.

In the methods provided herein, ascaroside derivatives can be directly applied to the plant and/or soil or can be formulated into a composition that can be applied to the plant and/or soil. As such, the present disclosure provides compositions that generally comprise at least one ascaroside derivative and one or more inert ingredients, e.g., one or more agronomically acceptable carriers. It is preferred that non-toxic carriers be used in the methods of the present disclosure.

The term “agronomically acceptable carrier” includes any carrier suitable for administration to a plant or soil, e.g., customary excipients in formulation techniques, such as used to form solutions (e.g., directly sprayable or dilutable solutions), emulsions, (e.g., emulsion concentrates and diluted emulsions), wettable powders, suspensions, soluble powders, powders, dusts, pastes, soluble powders, granules, suspension-emulsion concentrates, encapsulation into polymeric materials, coatable pastes, natural and synthetic materials impregnated with active compound and microencapsulations in polymeric substances. These compositions can be produced in a known manner, for example, by mixing the ascaroside

(s) with one or more agronomically acceptable carriers, such as liquid solvents or solid carriers, optionally with the use of additional components including, but not limited to, surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, wetting agents, and the like.

In some embodiments, the ascaroside derivative is the only active agent within the composition. In some embodiments, the composition includes one or more additional ascarosides (e.g., an ascaroside or another ascaroside derivative). In some embodiments, one or more other active agents are included within the composition (e.g., one or more pesticides, fungicides, antibacterial compounds, herbicides fertilizers, etc.). The ascaroside derivative-containing compositions provided herein can be in various forms, including solid and liquid forms.

If the agronomically acceptable carrier is water, in some embodiments, an organic solvent may be incorporated as an auxiliary liquid solvent. Suitable liquid solvents include, for example, aromatics (e.g., xylene, toluene and alkylnaphthalenes); chlorinated aromatics or chlorinated aliphatic hydrocarbons (e.g., chlorobenzenes, chloroethylenes and methylene chloride); aliphatic hydrocarbons (e.g., cyclohexane); paraffins (e.g., petroleum fractions, mineral and vegetable oils); alcohols (e.g., ethanol, butanol, glycol, propylene glycol and their ethers and esters); ketones (e.g., acetone, methyl ethyl ketone, methyl isobutyl ketone and cyclohexanone) and strongly polar solvents (e.g., dimethylformamide, acetonitrile and dimethyl sulfoxide).

Suitable solid agronomically acceptable carriers include, for example, ammonium salts and ground natural minerals (e.g., kaolins, clays, talc, chalk, quartz, attapulgite, montmorillonite and diatomaceous earth); ground synthetic minerals (e.g., highly disperse silica, alumina and silicates); crushed and fractionated natural rocks (e.g., calcite, marble, pumice, sepiolite and dolomite); synthetic granules of inorganic and organic meals; granules of organic material (e.g., sawdust, coconut shells, maize cobs and tobacco stalks).

Suitable emulsifiers and foam-formers include, for example, nonionic and anionic emulsifiers (e.g., polyoxyethylene fatty acid esters, polyoxyethylene fatty alcohol ethers, for example, alkylaryl polyglycol ethers, alkylsulfonates, alkyl sulphates and arylsulfonates) protein hydrolysates.

Suitable dispersants include, for example, lignin-sulfite waste liquors and methylcellulose. Tackifiers such as carboxymethylcellulose and natural and synthetic polymers in the form of powders, granules or lattices, such as gum arabic, polyvinyl alcohol and polyvinyl acetate, as well as natural phospholipids, such as cephalins and lecithins, and synthetic phospholipids, can be used in the disclosed compositions. Other additives may include, for example, mineral and vegetable oils.

Colorants such as inorganic pigments, for example, iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc may also be included in the compositions.

Methods of preparing solid and liquid compositions for agrichemical use are generally known and can be employed according to the present disclosure (where such methods involve incorporating one or more ascaroside salts) within such compositions). Compositions according to the present disclosure can, in some embodiments, be in the form of granular material (including dusts, pellets, soluble powders, flowable powders, water-dispersible granules, and the like). In some embodiments, compositions according to the present disclosure can be in liquid form (e.g., solutions, suspensions, or emulsions). In some embodiments, compositions are in the form of a granular material treated with an ascaroside-derivative containing liquid. In some embodiments, a composition comprising an ascaroside derivative is formed into fibers or filaments and in some such embodiments, a woven or non-woven textile (e.g., film) can be produced therefrom. In some embodiments, a composition as provided herein is pelletized. In some embodiments, a composition as provided herein is in the form of a film, e.g., plastic mulch. Any of the solid compositions provided herein can optionally be coated via methods generally known in the art to delay release of the ascaroside derivative.

In certain embodiments, the ascaroside derivatives described herein and compositions containing such derivatives have utility for the treatment of diseases or disorders in animals including the treatment of human diseases and disorders. As such, in certain embodiments, the invention provides pharmaceutical compositions containing one or ascaroside derivatives as described herein. In certain embodiments, the ascaroside derivatives are useful to formulate compositions and to treat disorders as described in U.S. Pat. No. 11,077,151 the entirety of which is hereby incorporated herein by reference.

### III. EXAMPLES

Aspects of the present disclosure are more fully illustrated by the following examples, which are set forth to illustrate certain aspects of the present disclosure and are not to be construed as limiting thereof.

### Example 1. Synthesis of Ascaroside Analogs

Examples 1, 1a through 1, 1c demonstrate the syntheses of ascr #18 amides from the ascr #18 methyl ester (methyl 10-(((2R,3R,5R,6S)-3,5-dihydroxy-6-methyltetrahydro-2H-pyran-2-yl)oxy) decanoate) by reaction with primary or secondary amines. It will be appreciated that additional analogs can readily be produced with these methods by utilizing different amines, other methyl ascarosides, or other esters (e.g., ethyl, propyl, benzyl) of ascr #18. Scheme E1 shows the reagents and products of Examples 1a through 1c.

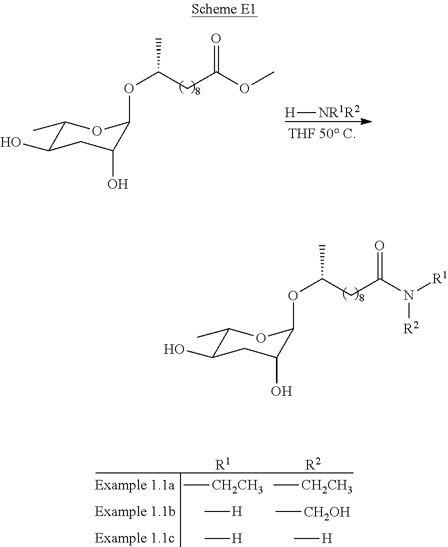

### Example 1.1a Synthesis of ascr #18 diethylamide ((R)-10-(((2R,3R,5R,6S)-3,5-dihydroxy-6-methyltetrahydro-2H-pyran-2-yl)oxy)-N,N-diethylundecanamide)

5.0 grams (15 mmol) of ascr #18 methyl ester was dissolved in anhydrous THF (20 mL). Diethylamine (4.1 mL, 40 mmol) was added in one portion. The mixture was heated to 50° C. and stirred for 60 hours. The mixture was cooled to room temperature and poured into separatory funnel containing 50 mL of 0.1M aqueous HCl and 100 mL ethyl acetate. After mixing thoroughly, the aqueous layer was discarded and the ethyl acetate extract was washed sequentially with 50 mL 0.1M HCl, 50 mL DI water, and 50 mL sat. aqueous NaHCO₃ and 50 mL brine. The mixture was concentrated to provide the target compound as a clear syrup.

### Example 1.1b Synthesis of ascr #18 ethanolamide ((R)-10-(((2R,3R,5R,6S)-3,5-dihydroxy-6-methyltetrahydro-2H-pyran-2-yl)oxy)-N-(2-hydroxyethyl)-undecanamide)

This compound was produced according to the method described in Example 1.1a, except ethanolamine was substituted for diethylamine.

### Example 1.1c Synthesis of ascr #18 carboxamide ((R)-10-(((2R,3R,5R,6S)-3,5-dihydroxy-6-methyltetrahydro-2H-pyran-2-yl)oxy)-undecanamide)

This compound was produced according to the method described in Example 1.1a, except 10 mL of an 8M solution of ammonia was substituted for the diethylamine and the mixture was heated for 96 h instead of 60 h.

Examples 1.1d through 1.1f demonstrate the synthesis of oscr #16 amides as shown in Scheme E2. These products are formed according to the methods of Examples 1a through 1c, in each case substituting oscr #16 methyl ester (methyl 10-(((2R,3R,5R,6S)-3,5-dihydroxy-6-methyltetrahydro-2H-pyran-2-yl)oxy) decanoate) for the ascr #18 methyl ester.

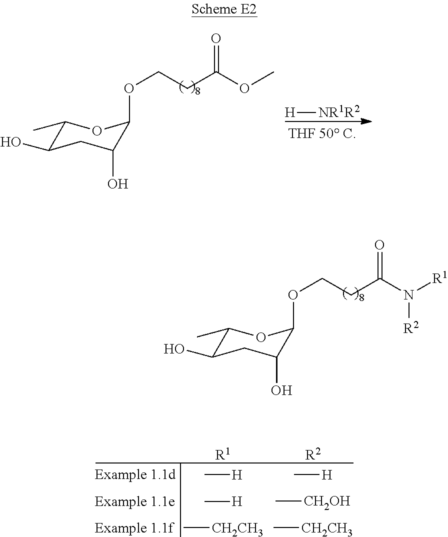

### Example 1.1g Demonstrates the Synthesis of an Amide-Linked Ascr #18 Dimer as Shown in Scheme E3

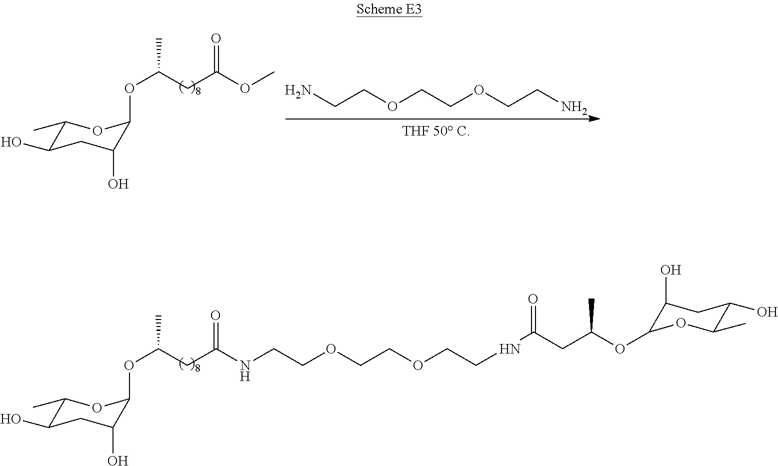

### Example 1.1g Synthesis of ascr #18-(ethylenedioxy)bis(ethylamine)

Dimer (10R)-10-(((2R,3R,5R,6S)-3,5-dihydroxy-6-methyltetrahydro-2H-pyran-2-yl)oxy)-N-(2-(2-(2-((10R)-10-(((3S,5S,6R)-3,5-dihydroxy-6-methyltetrahydro-2H-pyran-2-yl)oxy)undecanamido)ethoxy)ethoxy)ethyl)undecanamide).

5.0 grams (15 mmol) of ascr #18 methyl ester is dissolved in anhydrous THF (20 mL). 2,2′-(Ethylenedioxy)bis(ethylamine) (1.1 mL, 7.5 mmol) is added in one portion. The mixture is heated to 50° C. and stirred for 60 hours. The mixture is cooled to room temperature and poured into separatory funnel containing 50 mL of 0.1M aqueous HCl and 100 mL ethyl acetate. After mixing thoroughly, the aqueous layer is discarded and the ethyl acetate extract is washed sequentially with 50 mL 0.1M HCl, 50 mL DI water, and 50 mL sat. aqueous NaHCO₃ and 50 mL brine. The mixture is concentrated to provide the target compound.

### Example 2 Synthesis of Amidine Derivatives of Ascarosides

As shown in Scheme E4, ascr #18 methyl ester is treated according to the methods described in Tetrahedron Letters 43 (2002) 419-421, to provide the amidine of ascr #18 ((R)-10-(((2R,3R,5R,6S)-3,5-dihydroxy-6-methyltetrahydro-2H-pyran-2-yl)oxy)undecanimidamide).

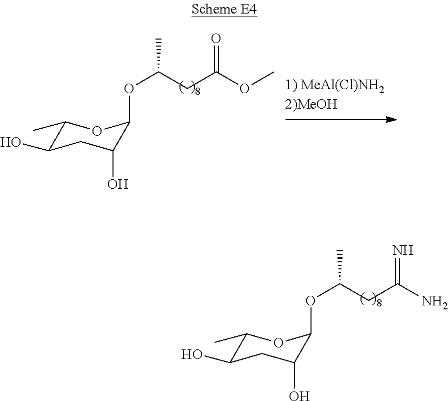

### Example 3. Ascaroside Analogs Enhance Resistance Against a Fungal Pathogen in Wheat Seedlings

The products of Examples 1, 1a to 1, 1c (hereafter referred to as compounds 1a, 1b, and 1c) were evaluated in a wheat pathogen assay to assess the potential of these analogs to prevent crop damage caused by a fungal pathogen. Wheat seeds of cv. Louise were planted in potting soil and grown for two weeks in a growth chamber at 26° C. and 23° C., respectively, with 16 hours of light. After two weeks, the plants were sprayed with a 1 μM solution of ascr #18, or compounds 1a, 1b, or 1c containing 0.1% (v/v) Tween®-20 in water solution, or with a mock solution only containing 0.1% (v/v) Tween®-20. 48 hours after treatment, the wheat plants were inoculated with a Bipolaris sorokiniana spore solution (with 15000 spores/ml) and placed in a dark grow tent with high humidity (>80% RH) for 24 hours at 23° C. Subsequently, the plants were moved to a high humidity environment (>80% RH) at 23° C. and 12 hours of light and dark until fungal infection was visible on the leaves of the mock-treated plants (usually after about four days). Symptoms were evaluated by photographing the infected leaves and processing the images through machine learning image analysis software (Ilastik) to determine the symptomatic areas relative to the total leaf area.

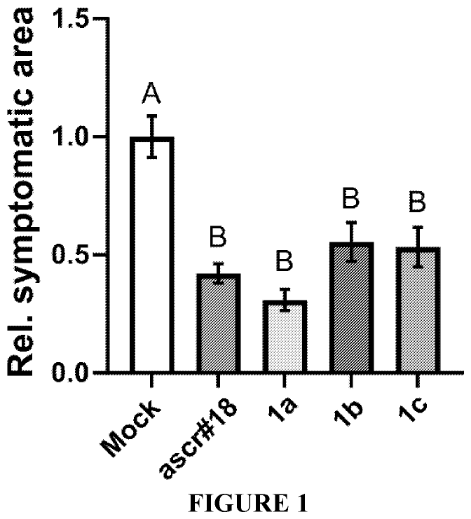

FIG. 1 shows a bar chart of the results. It can be seen that wheat plants that were treated with 1 μM solutions of compounds 1a, 1b, or 1c showed reduced infection symptoms compared to the plants that were treated with mock. The infection levels in the treated plants were similar to those treated with 1 μM ascr #18. This demonstrates that compounds 1a, 1b, and 1c are effective in reducing fungal infection in wheat, and that their efficacy is comparable to that of ascr #18 in this assay. In FIG. 1, error bars depict standard error of the mean for a total n≥35 plants across 4 independent experiments. Outliers were removed with the GraphPad ROUT method, and significance determined by a one-way ANOVA test.

### Example 4 Metabolism of Ascaroside Amides

Ascaroside analogs 1a, 1b, and 1c were also evaluated in alfalfa seedlings to assess whether plants take up these analogs and whether they are hydrolyzed or metabolized to other ascarosides (specifically to ascr #18 and/or ascr #18 metabolites). The ascr #18 metabolism was evaluated by assaying the plant tissues for the known ascaroside metabolite ascr #9.

Alfalfa seeds were germinated in the dark on culture plates (10 seeds per plate) containing Gelzan growth medium and were allowed to grow for 3-4 days before treatment. Treatment consisted of flooding the plates with 10 μM aqueous solutions of one of the ascaroside analogs 1a, 1b, or 1c, with ascr #18 (as a positive control), or with plain water as a mock treatment (5 ml total volume, solutions prepared in MQ water). Each experiment was performed in duplicate. After 24 hours, the plates were moved to an illuminated growth chamber for 4 hours before sampling.

Sprouts were collected, thoroughly rinsed with MQ water, dried, flash-frozen, and homogenized. The homogenized tissues were extracted with 100% ethanol and the extracts were analyzed using high-resolution liquid chromatography-mass spectrometry (LC-MS). LC-MS analysis was performed using a Dionex 3000 UPLC coupled with a Thermo Q Exactive high-resolution mass spectrometer equipped with a HESI ion source. To measure ascarosides, samples were chromatographed using an Agilent Zorbax Eclipse column (150 mm×2.1 mm, particle size 1.98 μm) maintained at 40° C. with a 0.5 ml/min flow rate. Solvent A: 0.1% formic acid in water; Solvent B: 0.1% formic acid in acetonitrile. A solvent mixture using 1% B was used for 1.5 min after injection, followed by a linear gradient up to 99% B at 9.5 min, followed by 0.75 min of 99% B, then back to 1% B over 0.5 min, and finally held at 1% B for an additional 1.25 min to re-equilibrate the column (total time: 12 min, with needle washing and file writing: ˜14 min). Mass spectrometer parameters: spray voltage (−3.0 kV or +3.5 kV); capillary temperature 380° C.; probe heater temperature 400° C.; sheath, auxiliary, and sweep gas 60, 20, and 2 AU, respectively—S-Lens RF level: 50, resolution 120,000 at m/z 200, AGC target 3E6. Samples were injected and analyzed in negative electrospray ionization mode with an m/z range of 100-1000. Ascr #18 (C17H32O6) ionizes in negative ion mode as C17H31O6, m/z=331.2126, retention time 6 minutes using the above chromatographic conditions. Samples were also analyzed in positive ionization mode.

The LCMS results are summarized in Table 1. Extracts from sprouts treated with ascr #18, as well as from those treated with ascaroside analogs 1b and 1c, show detectable levels of ascr #18 and/or its known metabolite ascr #9. However, neither ascr #18 nor ascr #9 was detected in the sprouts treated with analog 1a. The treatment solutions used to treat the sprouts were also analyzed assayed and the results are shown in the Column of Table 1 labeled “Input” In each case, only the analog, or ascr #18 respectively is detected, indicating the molecules are stable in the treatment solutions.

TABLE 1
LC-MS analysis of alfalfa tussues 24 h after treatment with ascr#18 or
provided ascaroside analogs. The values shown are the LCMS detector
response for diagnostic from each analog (Input) and for ascr#18 and
its metabolite ascr#9. These values represent the relative
abundance of ascr#18 and ascr#9 in the plant tussues.

| Sample | Repeat | Input | ascr#18 | ascr#9 |
|---|---|---|---|---|
| 1a | Rep1 | 6.78E+06 | ND | ND |
| 1a | Rep2 | 4.03E+06 | ND | ND |
| 1b | Rep1 | 3.46E+06 | 2.72E+05 | 3.23E+06 |
| 1b | Rep2 | 1.27E+06 | 1.14E+05 | 3.17E+06 |
| 1c | Rep1 | 5.65E+06 | ND | 8.81E+05 |
| 1c | Rep2 | 1.56E+06 | 1.14E+05 | 3.17E+06 |
| ascr#18 | Rep1 | 1.32E+06 | ND | 7.70E+06 |
| ascr#18 | Rep2 | 6.17E+05 | ND | 8.90E+06 |
| Mock | Rep1 | ND | ND | ND |
| Mock | Rep2 | ND | ND | ND |

ND = not detectable.

The data in Table 1 suggest that ascaroside analogs 1b and 1c are hydrolyzed or metabolized by the plant. The fact that ascr #18 is still detectable in the analog treated alfalfa plants, but not in plants treated with ascr #18 directly, suggests these analogs may have a longer half-life in the plant than ascr #18. Interestingly Ascaroside analog 1a is not hydrolyzed or metabolized by the alfalfa plants in the timeframe shown. In extracts from sprouts treated with this analog compound 1a was the only ascaroside detected. This indicates that this molecule is taken up by plants and that it has a longer half life than ascr #18 or the other analogs tested. These data when considered with the efficacy of analog 1a in the wheat pathogen assay described in Example 3, suggest that this analog may have itself have utility as a crop protection treatment (e.g., its efficacy in the pathogen assay may not be via hydrolysis or metabolism to ascr #18).

It is contemplated that compounds, compositions, and methods of the present application encompass variations and adaptations developed using information from the embodiments described in the present disclosure. Adaptation or modification of the methods and processes described in this specification may be performed by those of ordinary skill in the relevant art.

It will be appreciated that use of headers in the present disclosure are provided for the convenience of the reader. The presence and/or placement of a header is not intended to limit the scope of the subject matter described herein. Unless otherwise specified, embodiments located in one section of the application apply throughout the application to other embodiments, both singly and in combination.

Throughout the description, where compositions, compounds, or products are described as having, including, or comprising specific components, or where processes and methods are described as having, including, or comprising specific steps, it is contemplated that, additionally, there are articles, devices, and systems of the present application that consist essentially of, or consist of, the recited components, and that there are processes and methods according to the present application that consist essentially of, or consist of, the recited processing steps.

It should be understood that the order of steps or order for performing certain action is immaterial so long as the described method remains operable. Moreover, two or more steps or actions may be conducted simultaneously.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A composition comprising an ascaroside derivative of formula I: 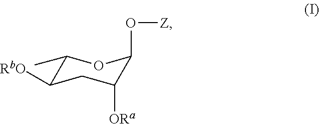
wherein:
each of R^{a} and R^{b} is independently —H, or an optionally substituted moiety selected from the group consisting of: C₁₋₂₀ aliphatic, C₁₋₂₀ acyl, C₁₋₂₀ heteroaliphatic, aryl, heteroaryl, a hydroxyl protecting group, a phosphorous-linked functional group, a sulfur-linked functional group, a silicon-linked functional group, a C₂₋₂₀ carbonate (e.g.—a moiety —C(O)OR^{c}), a C₂₋₂₀ carbamate (e.g.—a moiety —C(O)N(R^{c})₂), a C₂₋₂₀ thioester (e.g. a moiety —C(S)R^{c}), a C₂₋₂₀ thiocarbonate (e.g. a moiety —C(S)OR^{c}), a C₂₋₂₀ dithiocarbonate (e.g. a moiety —C(S)SR^{c}), a C₁₋₂₀ thiocarbamate (e.g. a moiety —C(S)N(R^{c})₂), a sugar moiety, a peptide, a polymer chain, or a linkage via a bond or a carbon-containing linker moiety to another ascaroside molecule, where Re is independently at each occurrence selected from —H, optionally substituted C₁₋₁₂ aliphatic, optionally substituted C₁₋₁₂ heteroaliphatic, optionally substituted aryl, optionally substituted heteroaryl, a polymer chain, or a linkage via a bond or a carbon-containing linker moiety to another ascaroside molecule; and where R^{a} and R^{b} may be taken together to form an optionally substituted ring, optionally containing one or more heteroatoms, and optionally containing one or more sites of unsaturation; and
—Z comprises an optionally unsaturated, optionally substituted C₂₋₄₀ sidechain terminating in a nitrogen- or sulfur-containing functional group.

2. The composition of claim 1, wherein —Z has a structure selected from the group consisting
of: —CH(CH₃)—(CH₂)ₙ—Q, —CH(CH₃)—(CH₂)ₙ—CH—CH—Q, —CH(CH₃)—(CH₂)ₙ—CH═CH—(CH₂)ₙ—Q, —CH(CH₃)—(CH₂)ₙ—CH(OH)—CH₂—Q, —CH(CH₃)—(CH₂)ₙ—CH(OH)—Q, —(CH₂)ₙ—Q, —(CH₂)ₙ—CH═CH—Q, —(CH₂)ₙ—CH═CH—(CH₂)ₙ—Q, —(CH₂)ₙ—CH(OH)—CH₂—Q, and —(CH₂)ₙ—CH(OH)—Q, where —Q is a nitrogen- or sulfur-containing functional group, and n is independently at each occurrence an integer between 1 and 40.

3. The composition of claim 2, wherein —Q is a nitrogen-containing functional group.

4. The composition of claim 3, wherein Q is selected from the group consisting of: amide, amidine, nitrile and imide.

5. The composition of claim 3, wherein —Q is selected from the group consisting of: —C(O)NR¹R², —C(NR⁴)NR¹R², —C(O)NR¹C(O)R³, and —C≡N, where:
at each occurrence, R¹, R², and R³ are selected independently from the group consisting of: —H, optionally substituted C₁₋₁₂ aliphatic, optionally substituted C₁₋₁₂ heteroaliphatic, optionally substituted aryl, optionally substituted heteroaryl, a polymer chain, and a linkage via a bond or a carbon-containing linker moiety to another ascaroside molecule; where R¹ and R², or R¹ and R³ may optionally be taken together to form an optionally substituted ring, optionally containing one or more heteroatoms and optionally containing one or more sites of unsaturation; and
R⁴ is selected from the group consisting of: —H, optionally substituted C₁₋₁₂ aliphatic, optionally substituted C₁₋₁₂ heteroaliphatic, optionally substituted aryl, optionally substituted heteroaryl, and a polymer chain, or a linkage via a bond or a carbon-containing linker moiety to another ascaroside molecule.

6. The composition of claim 2, wherein —Q is a sulfur-containing functional group.

7. The composition of claim 6, wherein Q is selected from the group consisting of: thioacid, thioester, dithioester, thioamide, and thioimide.

8. The composition of claim 6, wherein —Q is selected from the group consisting of: —C(S)OR¹, —C(O)—Sₓ—SR¹, —C(S)—Sₓ—SR¹, —C(S)NR¹R², —C(S)NR¹C(O)R³, —C(O)NR¹C(S)R³, and —C(S)NR¹C(S)R³, where: x is an integer between 0 and 6, and at each occurrence; and R¹, R², and R³ are selected independently from the group consisting of: —H, optionally substituted C₁₋₁₂ aliphatic, optionally substituted C₁₋₁₂ heteroaliphatic, optionally substituted aryl, optionally substituted heteroaryl, a polymer chain, and a linkage via a bond or a carbon-containing linker moiety to another ascaroside molecule; where R¹ and R², or R¹ and R³ may optionally be taken together to form an optionally substituted ring, optionally containing one or more heteroatoms and optionally containing one or more sites of unsaturation.

9. The composition of any of claims 1-8, further comprising one or more additional components selected from the group consisting of surfactants, including emulsifiers, dispersants, foam-formers, colorants, processing aids, lubricants, fillers, reinforcements, flame retardants, light stabilizers, ultraviolet radiation absorbers, weather stabilizers, plasticizers, release agents, perfumes, heat-retaining additives (e.g., silica), cross-linking agents, antioxidants, anti-foaming agents, buffers, pH modifiers, compatibility agents, drift control additives, extenders/stickers, tackifiers, plant penetrants, safeners, spreaders, and wetting agents.

10. A method of treating an organism with an ascaroside and providing contacting the organism with ascaroside derivatives containing a nitrogen- or sulfur-containing functional group, wherein the nitrogen- or sulfur-containing functional group is then hydrolyzed or metabolized to a carboxylic acid (or an ester or salt thereof).

11. The method of claim 10, wherein the organism is a plant.

12. The method of claim 10, wherein the organism is a human.
